(19) United States
(12) Patent Application Publication
(10) Pub. No.: US 2009/0074795 A1
(43) Pub. Date: Mar. 19, 2009

(54) Methods for addressing ocular diseases through interference with the wnt signaling pathway

(76) Inventor: Rafal A. Farjo, Oklahoma City, OK (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 12/283,471

(22) Filed: Sep. 12, 2008

Related U.S. Application Data
(60) Provisional application No. 60/972,743, filed on Sep. 14, 2007.

(51) Int. Cl. A61K 39/395; A61K 31/7105; A61P 27/02

(52) U.S. Cl. 424/172.1; 514/44

(57) ABSTRACT

Choroidal neovascularization (CNV) in age-related macular degeneration (AMD) is a leading cause of blindness. Very low-density lipoprotein receptor gene knockout (Vldlr^{-/-}) mice have been shown to develop subretinal neovascularization (NV) with an unknown mechanism. The present disclosure presents novel methods for addressing eye-disease states characterized by angiogenesis or neovascularization by inhibiting the wnt signal pathway. Inhibition of the LRP5/6 receptor by an agent, for example DKK1 or antibody, is shown to inhibit the wnt pathway effecting reduction in ocular neovascularization and angiogenesis.

### RELATED APPLICATIONS

This application claims the Paris Convention Priority of and incorporates by reference as if fully disclosed herein U.S. Provisional Patent Application Ser. No. 60/972,743 filed on Sep. 14, 2007.

### BACKGROUND

Blindness caused by the retinal and choroidal neovascularization (CNV) in diabetic retinopathy and in age-related macular degeneration (AMD) remains one of the most urgent medical problems of our times. The significance of these blinding disorders is growing as the population ages and the incidence of diabetes increases. The treatments for AMD are limited by the fact that relatively little is currently understood about the biochemical pathways or pathogenic mechanisms underpinning AMD and consequent CNV.

The retina is supported by two separate vascular networks: the retinal vessels and the choroidal vessels. There are two major steps in vascular development, vessel formation, and vessel maturation. Vessel formation mainly involves endothelial proliferation and migration, forming pericyte-free retinal vasculature. Vascular endothelial growth factor (VEGF) is known to play a key growth-promoting role in vessel formation.

Wnts are a group of secreted, cysteine-rich glycoproteins that bind to frizzled (Fz) receptors or to Fz/LDL receptor-related protein 5 or 6 (LRP5/6) co-receptors and regulate expression of a number of target genes. In the absence of wnts, transcription factor β-catenin is phosphorylated by a protein complex containing glycogen synthase kinase-3β (GSK-3β). The phosphorylated β-catenin is continuously degraded. Upon binding of certain wnts to the Fz-LRP5/6 co-receptors, phosphorylation of β-catenin is inhibited, which decreases the degradation of β-catenin and results in its accumulation. β-catenin is then translocated into the nucleus and regulates expression of target genes including VEGF.

Recent evidence has demonstrated that the wnt signaling pathway plays a role in the regulation of angiogenesis. Mutations in the Fz4 or LRP5 gene in the human have been found to inhibit the normal retinal angiogenesis in the familial exudative vitreoretinopathy (FEVR) patients, while Fz4 knockout (fz4^{-/-}) mice exhibited an incomplete retinal vascularization. Moreover, Norrin, a downstream binding ligand of the Fz4 receptor, is associated with the vascular development in the eye, as mutations in, or complete knockout of this gene, as found in Norrie disease, results in congenital absence of intra-retinal capillaries and a progressive loss of vessels within the stria vascular is in the cochlea. Meanwhile, the key protein in the wnt signaling pathway, β-catenin has also been implicated in vascular development and remodeling. It has been shown that the membrane pool of β-catenin is required for mitogenic signaling through the VEGF receptor-2 (VEGFR2)-mediated activation of PI3-kinase and Akt.

Very low-density lipoprotein receptor gene knockout (Vldlr^{-/-}) mice were initially created to study the cholesterol pathway. However, Vldlr^{-/-} mice have been shown to lack dyslipidemia, and as such are of little use in cholesterol research. In a comprehensive ocular phenotype screen, it was discovered through fundus examination that Vldlr^{-/-} mice develop abnormal subretinal NV. To date, the underlying mechanism by which disruption of the Vldlr gene (knockout) leads to the subretinal NV has not been elucidated. The present disclosure is an examination of the role of the wnt signaling pathways in the subretinal NV in Vldlr^{-/-} mice, and presents methods for treating eye-related diseases by interfering with the wnt signaling pathways.

### SUMMARY

Choroidal neovascularization (CNV) in age-related macular degeneration (AMD) is a leading cause of blindness. Very low-density lipoprotein receptor gene knockout (Vldlr^{-/-}) mice have been shown to develop subretinal neovascularization (NV) with an unknown mechanism. The present disclosure presents novel methods for addressing eye-disease states characterized by angiogenesis or neovascularization by inhibiting the wnt signal pathway. Inhibition of the LRP5/6 receptor by an agent, for example DKK1 or antibody, is shown to inhibit the wnt pathway effecting reduction in ocular neovascularization and angiogenesis.

According to a feature of the present disclosure, a method is disclosed comprising treating an animal having an eye-related disease with a DKK-related composition. A symptom of the eye-related disease is at least one of angiogenesis and neovascularization.

According to a feature of the present disclosure, a method is disclosed comprising treating an animal having an eye-related disease with an effective amount of an antibody directed against a LRP5/6 receptor. A symptom of the eye-related disease is at least one of angiogenesis and neovascularization.

According to a feature of the present disclosure, a method is disclosed comprising treating an eye-related disease by administering an agent that interferes with the wnt pathway effecting increased phosphorylation of β-catenin. A symptom of the eye-related disease is at least one of angiogenesis and neovascularization.

According to a feature of the present disclosure, a method is disclosed comprising treating an eye-related disease by administering an agent that modulates LRP5/6 activity by preventing binding of molecules other than the agent to the LRP5/6 receptor. A symptom of the eye-related disease is at least one of angiogenesis and neovascularization.

### DRAWINGS

The patent or application file contains at least one drawing executed in color. Copies of this patent application publication with color drawing(s) will be provided by the Office upon request and payment of the necessary fee.

The above-mentioned features and objects of the present disclosure will become more apparent with reference to the following description taken in conjunction with the accompanying drawings wherein like reference numerals denote like elements and in which:

FIG. 1 is microscopy photographs an embodiment of experimental data showing subretinal neovascularization (NV) in the Vldlr^{-/-} eye;

FIG. 2 are microscopy photographs, western blot autoradiographs, and RT-PCR graphs of embodiments of experimental data showing increased VEGF and VEGFR2 levels in Vldlr^{-/-} eyecups;

FIG. 3 are microscopy photographs of embodiments of experimental data showing altered polarization of VEGF distribution in RPE cells in the Vldlr^{-/-} mice;

FIG. 4 are microscopy photographs, graphs of results of RT-PCR experiments, and western blot analysis of embodiments of experimental data showing elevated levels of wnt co-receptor-LRP5/6 in the Vldlr^{-/-} mouse eyecup;

FIG. 5 are western blot autoradiographs and RT-PCR graphs of embodiments of experimental data showing diminished phosphorylation of GSK-3β in the Vldlr^{-/-} eyecup;

FIG. 6 are western blot autoradiographs and RT-PCR graphs of embodiments of experimental data showing abolished phosphorylation of β-catenin in Vldlr^{-/-} eyecups;

FIG. 7 are embodiments of experimental data of western blot autoradiographs, microscopy photographs, and results from small interfering RNA experiments showing up-regulation of LRP5/6 expression by the VLDLR siRNA in endothelial cells;

FIG. 8 are photographs of embodiments of experimental data showing activation of β-catenin by the VLDLR siRNA in endothelial cells;

FIG. 9 are embodiments of experimental data comprising western blot autoradiographs, graphs of RT-PCT results, immunohistochemistry microscopy photographs, and results from small interfering RNA experiments demonstrating inhibition of the wnt signaling and VEGF expression by DKK1;

FIG. 10 are photographs of embodiments of experimental data comprising activated wnt signaling in the human retina with diabetic retinopathy;

FIG. 11 are photographs of embodiments of experimental data comprising western blot data showing elevated β-catenin levels in the retina of Akita mice;

FIG. 12 are photographs of embodiments of experimental data suggesting that oxidative stress is responsible for the wnt pathway activation by high glucose in bovine retinal capillary endothelial cells (BRCEC);

FIG. 13 is a graph of embodiments of experimental data suggesting that DKK1 down-regulates ICAM-1 expression in the retina of diabetic rats;

FIG. 14 is a graph of embodiments of experimental data suggesting DKK1 inhibits reactive oxygen species (ROS) generation induced by high glucose or TNF-α;

FIG. 15 is a photograph of embodiments of experimental data of western blots suggesting increased retinal β-catenin levels in rats with STZ-induced diabetes;

FIG. 16 is a photograph of embodiments of experimental data of showing activation of wnt pathway by hypoxia;

FIG. 17 are photographs of embodiments of experimental data showing the effects of DKK1on HIF-1 activation and VEGF over-expression induced by the wnt pathway;

FIG. 18 are photographs of embodiments of experimental data showing the effects of DKK1on VEGF expression in the retina of OIR rats;

FIG. 19 are photographs of embodiments of experimental data showing the inhibitory effect of DKK1on retinal neovascularization;

FIG. 20 are photographs of embodiments of experimental data showing the effect of DKK1on pre-retinal NV and HIF-1α levels in OIR retina; and

FIG. 21 is a graph of embodiments of experimental data showing the effect of DKK1 on retinal vascular permeability in the retina of STZ-induced diabetic rats.

### DETAILED DESCRIPTION

In the following detailed description of embodiments of the invention, reference is made to the accompanying drawings in which like references indicate similar elements, and in which is shown by way of illustration specific embodiments in which the invention may be practiced. These embodiments are described in sufficient detail to enable those skilled in the art to practice the invention, and it is to be understood that other embodiments may be utilized and that logical, mechanical, biological, electrical, functional, and other changes may be made without departing from the scope of the present invention. The following detailed description is, therefore, not to be taken in a limiting sense, and the scope of the present invention is defined only by the appended claims. As used in the present disclosure, the term “or” shall be understood to be defined as a logical disjunction and shall not indicate an exclusive disjunction unless expressly indicated as such or notated as “xor.”

As used herein, the term “eye-related disease” shall mean diseases of the eye characterized by at least angiogenesis or neovascularization.

Age-related macular degeneration is expressly contemplated as falling within the definition of “eye-related disease.” Other diseases include ocular disease characterized by over-active wnt pathway signaling, or overexpression of LRP5 or LRP6n, diabetic retinopathy, diabetic macular edema, retinitis, and uveitis. As used herein, the term “agent” shall mean a compound that has a beneficial effect in treating an eye-related disease.

As used herein, the term “effective amount” shall mean an amount of agent administered that effects a beneficial effect in treating an eye-related disease in an animal.

Angiogenesis in ocular tissues is a delicately coordinated process. Abnormal angiogenesis (neovascularization) in the retina or subretinal space is a common cause of vision loss in a number of ocular disorders, but its pathogenesis is not fully understood. The present study demonstrates that Vldlr knockout results in up-regulation of LRP5/6 expression in the retina and RPE and abnormal activation of the wnt signaling pathway, which mediates the over-expression of VEGF and CNV in Vldlr^{-/-} mice. Further, down-regulation of VLDLR alone by siRNA results in activation of the wnt signaling and VEGF over-expression, which can be blocked by a specific inhibitor of the wnt pathway. These observations for the first time established the function of VLDLR as a negative regulator of the wnt signaling pathway and revealed a role of VLDLR in the regulation of angiogenesis.

It was reported previously that Vldlr^{-/-} mice develop subretinal NV. The mechanism for the subretinal NV has not been investigated. The present study has demonstrated that the subretinal vasculature becomes a connected vascular network fusing the inner retina and choroidal vessels. As early as postnatal day 12, choroidal vessels begin to penetrate Bruch's membrane and the RPE layer, prior to retinal NV, suggesting that NV originates from the choroid in this mouse model. Moreover, NV progresses continuously in the sub-retinal space. These observations suggest that the Vldlr^{-/-} mouse is a CNV model.

VEGF is known as a major angiogenic factor, promoting normal and abnormal angiogenesis (11). RPE cells are known to express and secrete high levels of VEGF. Moreover, it is known that secretion of VEGF from the RPE cells are polarized, i.e., RPE cells secrete VEGF toward the choroid, which is proposed to be essential for maintaining the fenestration status of choroidal vessels. In the Vldlr^{-/-} RPE, the polarized secretion of VEGF is also disturbed as immunohistochemistry showed that some of VEGF signals were distributed near the surface adjacent to photoreceptors. The possible secretion of VEGF toward the photoreceptor layer in the Vldlr^{-/-} mice may contribute to subretinal NV. This is supported by the results showing that blocking VEGFR2 by subretinal injection of the neutralizing antibody inhibits subretinal NV.

Recent studies have established the role of the wnt signal in the regulation of angiogenesis. However, the role of the wnt signaling in CNV has not been investigated. Our results showed that Vldlr^{-/-} mice have increased levels of LRP5/6, a co-receptor of wnts, and abolished phosphorylation of GSK-3β. Further, phosphorylation of β-catenin, a downstream effector of the wnt signaling pathway, is also abolished in Vldlr^{-/-} eyecups, which resulted in stabilization and accumulation of β-catenin. Our results showed that silencing of VLDLR expression by siRNA results in increased LRP5/6 levels and β-catenin phosphorylation in cultured cells. These findings indicate that VLDLR gene knockout is a causative factor of the wnt signaling activation.

VLDLR is a member of the LDL receptor gene family. Unlike the LDL receptor, however, VLDLR has a widespread expression in many tissues. VLDLR is well known for its role in lipoprotein uptake and metabolism. The present study is the first to reveal its potential role in angiogenesis regulation. The results that knockdown of VLDLR by siRNA resulted in VEGF over-expression provides a direct evidence supporting the causative role of VLDLR deficiency in CNV.

To determine if the activation of the wnt signaling pathway mediates the CNV in Vldlr^{-/-} mice, we have used a DKK1, a wnt antagonist acting through specifically binding to the Frizzled co-receptors LRP5/6. The results indicated that DKK1 effectively blocked VEGF over-expression in the Vldlr^{-/-} eyecups and attenuated VEGF over-expression induced by the VLDLR siRNA in cultured cells. These experiments indicate that VEGF over-expression induced by VLDLR deficiency is mediated through the wnt pathway.

With respect to DKK1, the following U.S. patents and patent application publications are hereby entirely incorporated by reference: U.S. Pat. No. 6,844,422; U.S. patent Pub No. US 2008/0038775; and U.S. patent Pub. No. US 2003/0068312. Both of VLDLR and LRP5/6 belong to the LDL receptor gene family, but the interactions between VLDLR and LRP5/6 have not been reported previously. Several ligands for VLDLR have been identified previously, including plasminogen inhibitor type 1 (PAI-1), thrombospondin-1 (TSP-1) and tissue factor pathway inhibitor (TFPI). Further, VLDLR has been shown to mediate VLDL-induced PAI-1 gene transcription via regulating a transcription factor, namely VLDL-inducible factor-1. Our results that LRP5 and LRP6 mRNA levels are increased in Vldlr^{-/-} eyecups suggest that VLDLR regulates LRP5/6 gene expression, possibly at the transcriptional level. These findings suggest that VLDLR may be also coupled with some intracellular signaling pathways, through which, VLDLR regulates expression of target genes such as LRP5/6, in addition to its function in lipoprotein metabolism.

A recent genetic study reported a possible association of variations in the VLDLR, LRP6 and VEGF genes with AMD in human patients, suggesting a possible functional linkage between VLDLR and LRP5/6 in the retina and RPE. Further, the pattern of CNV in the Vldlr^{-/-} mouse recapitulates what is seen in wet AMD. Based on this notion, the Vldlr^{-/-} mouse may be considered a model of CNV associated with AMD.

According to embodiments, methods are disclosed for interfering with the wnt signaling pathway to address eye diseases characterized by angiogenesis and neovascularization. According to embodiments, such interference is accomplished by preventing binding of LRP5/6 or modulating LRP5/6 activity in the eye with ligands such as antibodies or DKK-related compositions (DKK molecules and analogs, e.g., DKK1 and DKK1 analogs), for example. Development of antibodies against LRP5/6 is well known in the art and can be accomplished without undue experimentation.

According to embodiments, a method of modulating ocular neovascularization, inflammation, and angiogenesis is presented. DKK1 is administered to an animal having symptoms or predisposition to ocular diseases characterized by neovascularization, inflammation, and angiogenesis, such as diabetes, age-related macular degeneration, ocular disease characterized by over-active wnt pathway signaling, or overexpression of LRP5 or LRP6, diabetic retinopathy, diabetic macular edema, retinitis, and uveitis. The Examples presented before supports the efficacy of DKK1 as an agent to modulate eye diseases.

According to embodiments, expression levels of LRP5/6 are modulated according to methods known and understood by artisans. As shown in the Examples, a significant increase of LRP5/6 mRNA and protein in Vldlr^{-/-} mice is observed. Therefore, according to embodiments, a method to treat eye-related diseases is to reduce the expression levels (e.g., restore to wild-type levels) of LRP5/6, as well as other receptors and molecules in the wnt signaling pathway. For example, small interfering RNA may be used in a therapeutic manner to reduce the level of LRP5/6 expression in animals having an eye-related disease, thereby reducing angiogenesis and neovascularization. SiRNA may be used to knock down VLDLR or LRP5/6 mRNA transcripts. Similarly, other small molecules and antibodies may be used to interfere with the LRP5/6 pathway. Artisans will readily understand how to produce antibodies, siRNA, antisense DNA, and other methods for interfering with the expression of genes and gene products in the wnt pathway.

According to another aspect, the agents of the present disclosure can be included in a pharmaceutical or nutraceutical composition together with additional active agents, carriers, vehicles, excipients, or auxiliary agents identifiable by a person skilled in the art upon reading of the present disclosure and administered to at least the eye of an animal, such as human.

The pharmaceutical or nutraceutical compositions preferably comprise at least one pharmaceutically acceptable carrier. In such pharmaceutical compositions, the agents of the present disclosure are the “active compound,” also referred to as the “active agent.” As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Subject as used herein refers to humans and non-human primates (e.g., guerilla, macaque, marmoset), livestock animals (e.g., sheep, cow, horse, donkey, and pig), companion animals (e.g., dog, cat), laboratory test animals (e.g., mouse, rabbit, rat, guinea pig, hamster), captive wild animals (e.g., fox, deer), and any other organisms who can benefit from the agents of the present disclosure. There is no limitation on the type of animal that could benefit from the presently described agents. A subject regardless of whether it is a human or non-human organism may be referred to as a patient, individual, animal, host, or recipient.

Pharmaceutical compositions suitable for an injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL.TM. (BASF, Parsippany, N.J.), or phosphate buffered saline (PBS). In all cases, the composition should be sterile and should be fluid to the extent that easy syringability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying, which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be transmucosal or transdermal. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

According to embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to cell-specific antigens) can also be used as pharmaceutically acceptable carriers. These can be prepared according to methods known to those skilled in the art, for example, as described in U.S. Pat. No. 4,522,811, which is incorporated by reference herein.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects can be used, care should be taken to design a delivery system that targets such compounds to the site of affected location to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of the active compound (i.e., an effective dosage) may range from about 0.001 to 100 g/kg body weight, or other ranges that would be apparent and understood by artisans without undue experimentation. The skilled artisan will appreciate that certain factors can influence the dosage and timing required to effectively treat a subject, including but not limited to the severity of the disease or disorder, previous treatments, the general health or age of the subject, and other diseases present.

According to another aspect, one or more kits of parts can be envisioned by the person skilled in the art, the kits of parts to perform at least one of the methods herein disclosed, the kit of parts comprising two or more compositions, the compositions comprising alone or in combination an effective amount of the agents of the present disclosure according to the at least one of the above mentioned methods.

The kits possibly include also compositions comprising active agents other than DKK1, identifiers of a biological event, or other compounds identifiable by a person skilled upon reading of the present disclosure. The term “identifier” refers to a molecule, metabolite or other compound, such as antibodies, DNA or RNA oligonucleotides, able to discover or determine the existence, presence, or fact of or otherwise detect a biological event under procedures identifiable by a person skilled in the art; exemplary identifiers are antibodies, exemplary procedures are western blot, nitrite assay and RT-PCR, or other procedures as described in the Examples. Exemplary biological events are cytokine expression or other immunomodulating events.

The kit can also comprise at least one composition comprising an effective amount the agents of the present disclosure or a cell line. The compositions and the cell line of the kits of parts to be used to perform the at least one method herein disclosed according to procedure identifiable by a person skilled in the art.

### EXAMPLES

### 1. CNV in Vldlr^{-/-} Mice

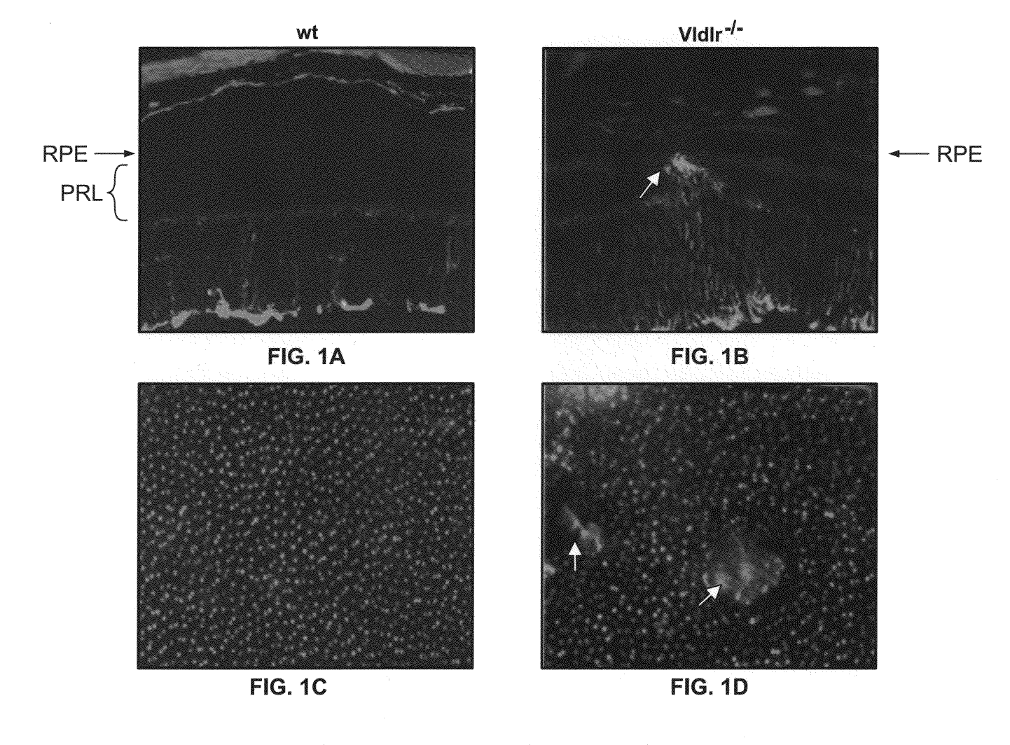

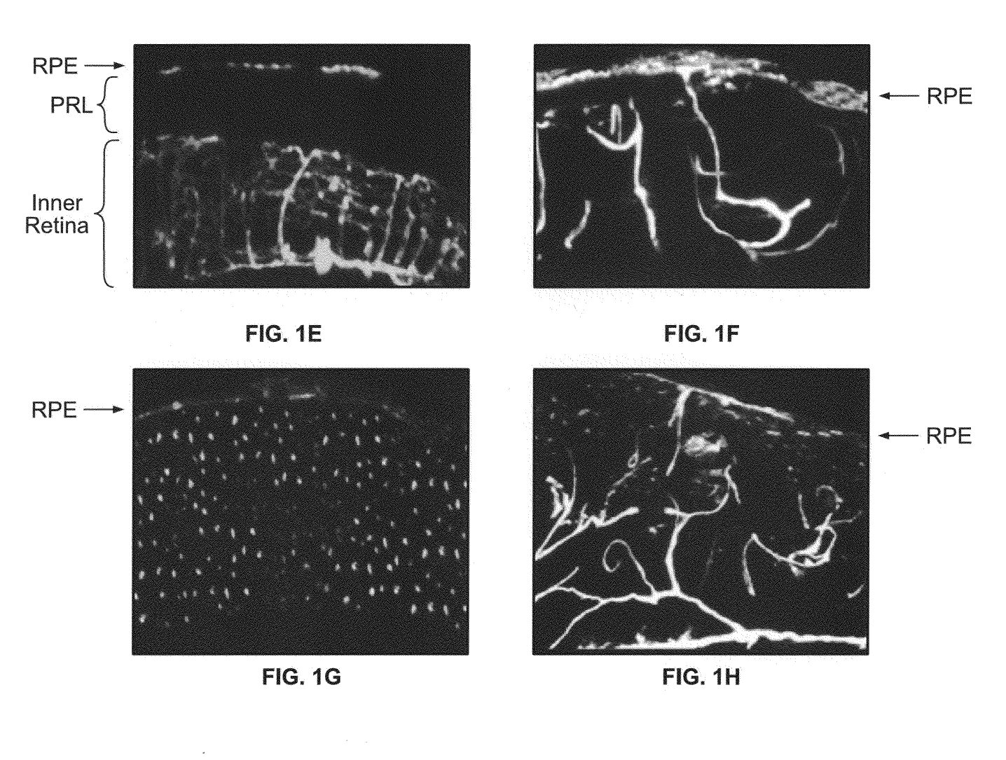

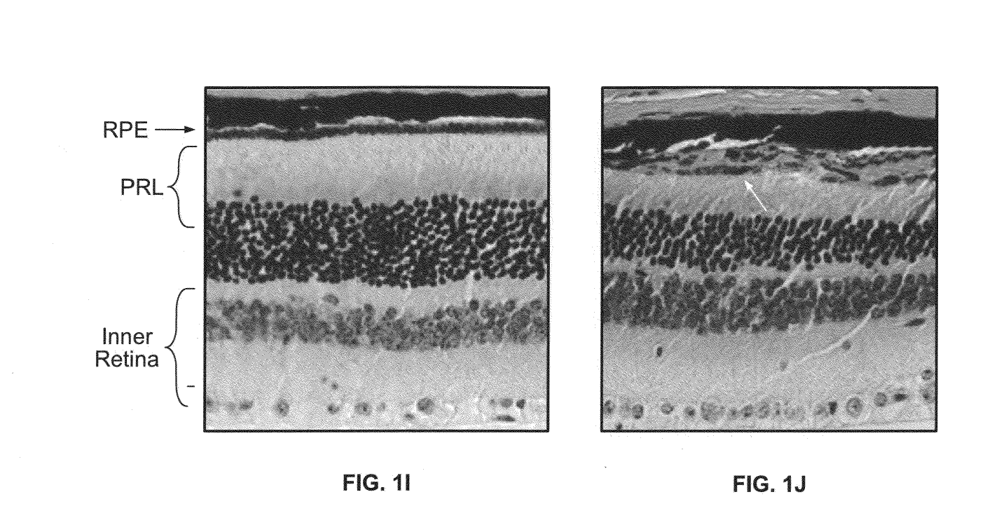

Vasculature in the retina and choroid in Vldlr^{-/-} and wt mice at ages of postnatal day 12 (P12) and 6 wks were analyzed by both in vivo vessel staining and fluorescein-angiography with FITC-conjugated high molecular weight dextran. In the retina of Vldlr^{-/-} mice at P12, retinal glial cells were observed to spread into the outer retina, as visualized by GFAP (a Müller cell marker) staining, while in wt mice, the GFAP signal was only detected in the inner retina, as shown in FIG. 1.

According to embodiments of experimental data, FIGS. 1A and 1B show data obtained where cross eye sections from wt (FIG. 1A) and Vldlr^{-/-} (FIG. 1B) mice (age P12) were double stained with an anti-GFAP antibody (green) and an anti-smooth muscle actin antibody (red). Note that although the neovasculature was not yet formed in Vldlr^{-/-} retina at P12, the glial cells (green) had spread into the photoreceptor layer (PRL) (indicated by an arrow) at this age. According to embodiments shown in FIGS. 1C and 1D, choroidal-RPE flat mounts following intravascular filling with fluorescein-conjugated high molecular weight dextran at age of P12. Unlike wt (FIG. 1C) which has an intact RPE layer, the neovasculature already penetrated from the choroid through Bruch's membrane and the RPE (indicated by arrows in FIG. 1D) in Vldlr^{-/-} mice. Note that the small dots in the RPE cell layer are due to autofluorescence of RPE cell nuclei. According to embodiments shown in FIGS. 1E-1H, thick retinal cross sections (200 μm) following in vivo vessel staining with an FITC-conjugated antibody against collagen IV to visualize the vascular network (RPE showed some auto-fluorescence) at age of 6 wks. As demonstrated by embodiments shown in Fig. E, no vasculature was detected in PRL in wt mice. According to embodiments shown if FIG. 1F, in the Vldlr^{-/-} retina, perpendicular vessels penetrated from the choroid to PRL. FIGS. 1G and 1H show images of wt and Vldlr^{-/-} retina and RPE, respectively taken at an angle of 15 degrees from the plane of the RPE. Unlike the absence of vessels at this area in the wt retina, the subretinal vascular network has formed clear connections with the choroidal vasculature in Vldlr^{-/-} mice at 6 wks of age. According to embodiments shown in FIGS. 1I and 1J, cross sections with hematoxylin and eosin (H&E) staining show accumulated neovasculature in the subretinal space in aged Vldlr^{-/-} mice (7 months of age) (FIG. 1J) but not in age-matched wt control. The arrow indicates the neovasculature in the subretinal space.

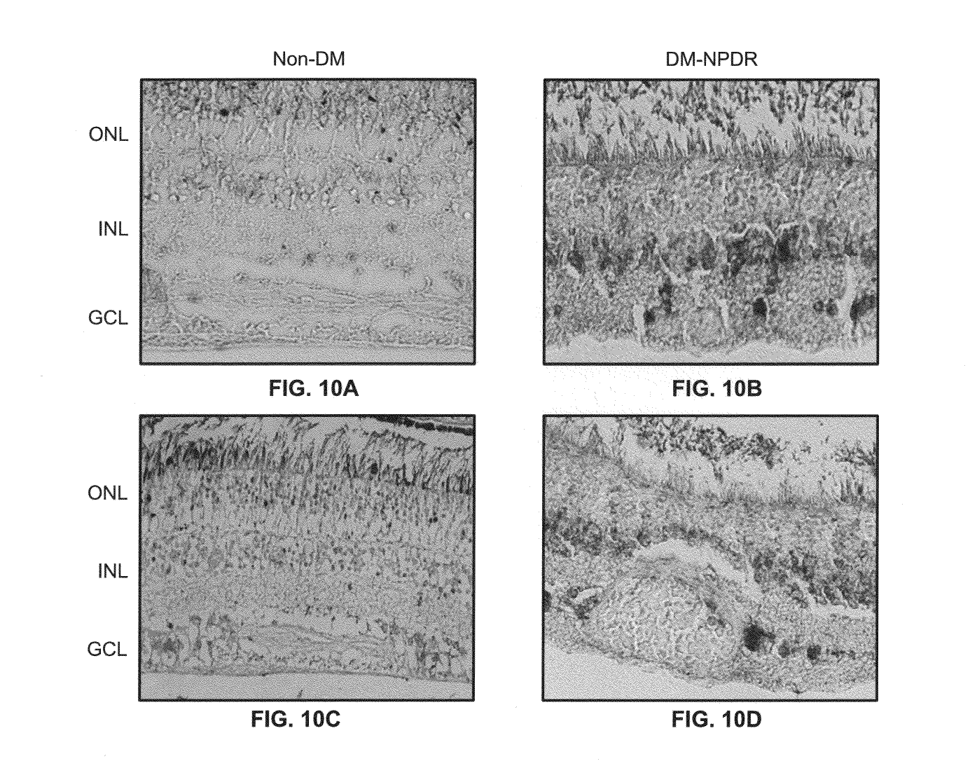

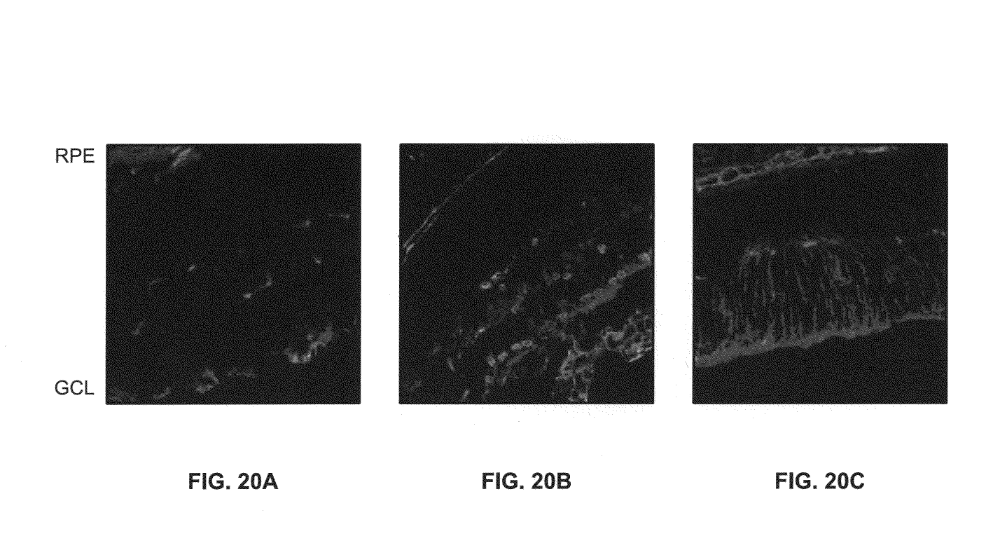

Immunostaining of CD31 (an endothelial marker) demonstrated no detectable retinal neovasculature in Vldlr^{-/-} mice at age of P12, but GFAP staining clearly demonstrated that abnormally increased GFAP-positive cells spread to the outer retina in Vldlr^{-/-} mice (FIGS. 1A and 1B). Concomitantly at this age, choroidal neovasculature can be seen to have penetrated through Bruch's membrane and the RPE from the choroid (FIG. 1D), in contrast to the preserved intact RPE layer in wt mice (FIG. 1C), suggesting that the NV is originated from the choroid in this model. At 6 wks of age, Vldlr^{-/-} mice showed abnormal NV throughout the subretinal space and the photoreceptor layer (FIGS. 1E-1H), consistent with the previous observations by Heckenlively et al. (32). By 6 wks of age, the choroidal vascular network has anastomosed with the retinal vasculature (FIGS. 1F and 1H). In older Vldlr^{-/-} mice (7 months of age), vasculature accumulated in the subretinal space, similar to the CNV in wet AMD (FIGS. 1I and 1J). The following scale is used in FIG. 1: 100 μm in (FIGS. 1C, 1D), 20 μm in (FIGS. 1A, 1B, 1E, 1F, 1I, and 1J), and 10 Mm in (FIGS. 1G, 1H).

### 2. Over-Expression of VEGF and VEGFR2 in Vldlr^{-/-} Evecups

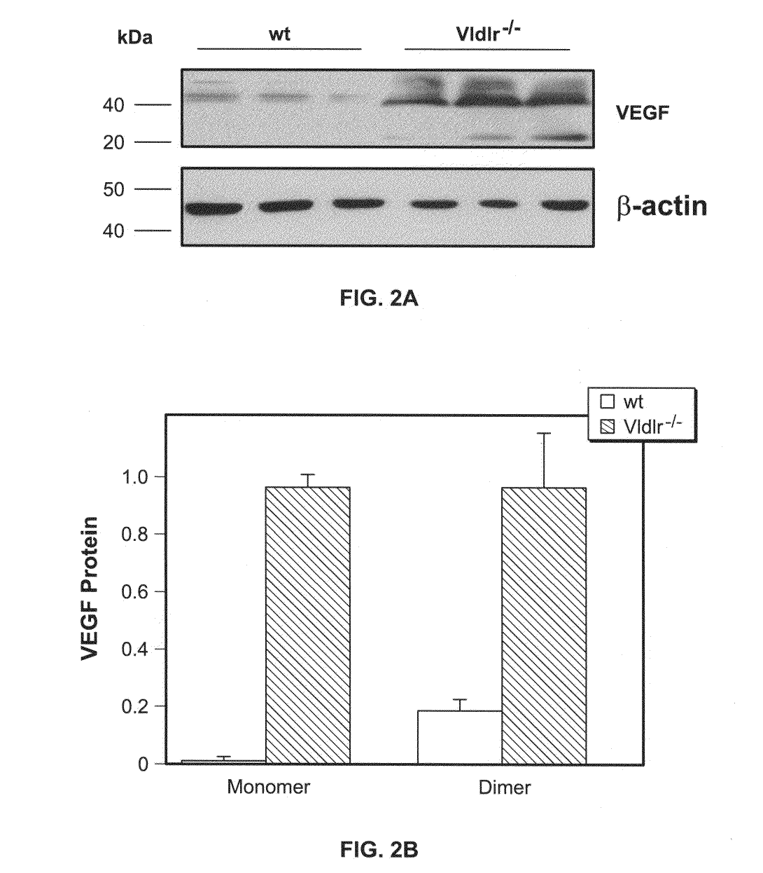

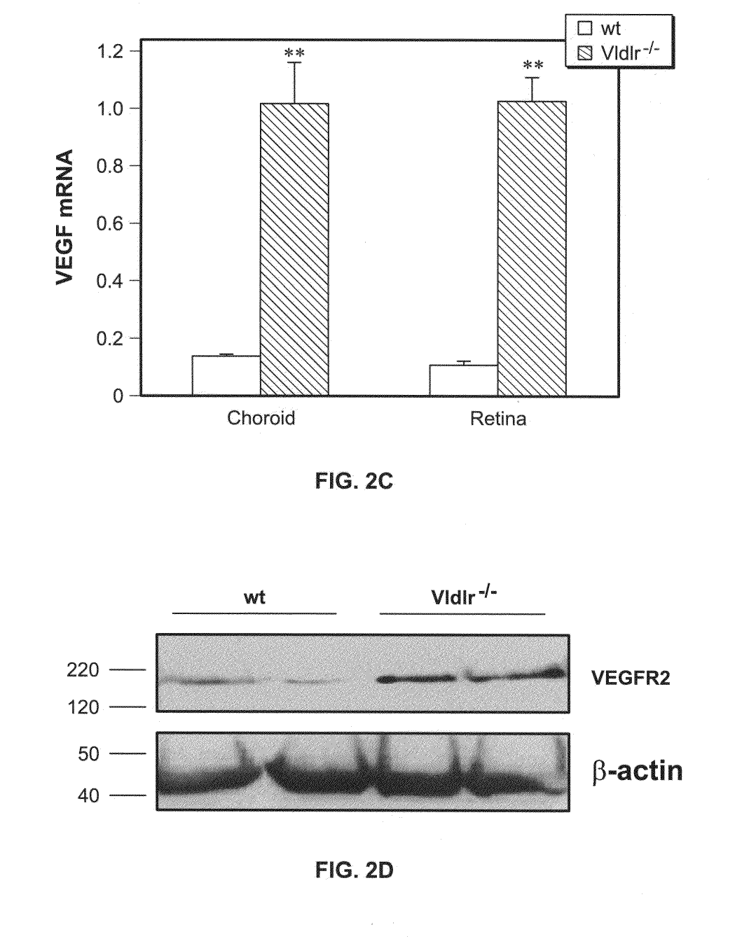

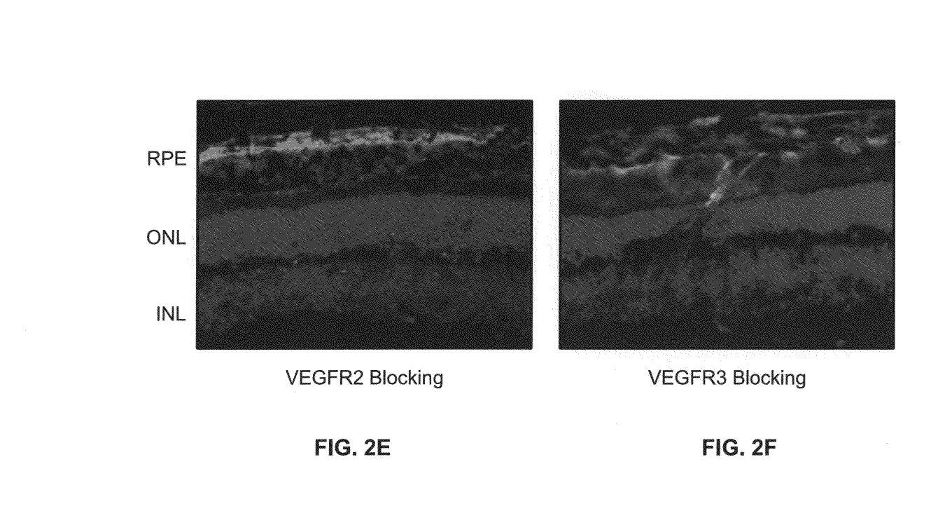

As VEGF is widely considered the major angiogenic factor in the retina, VEGF expression in the eyecups of Vldlr^{-/-} mice with those in the age-matched wt mice at both the protein and mRNA levels were compared. Western blot analysis demonstrated significantly elevated levels of the VEGF monomer and dimer in Vldlr^{-/-} eyecups in comparison to the wt eyecups (FIGS. 2A and 2B). Real-time RT-PCR showed that the increased VEGF expression occurs at the mRNA level in Vldlr^{-/-} mice (FIG. 2C). Moreover, VEGFR2 levels were also elevated in the Vldlr^{-/-} eyecups (FIG. 2D).

To provide additional evidence for the causative role of VEGF over-expression in the CNV in Vldlr^{-/-} mice, neutralizing antibodies (10 μg/eye) specific for VEGFR2 and VEGFR3 were injected separately into the subretinal space of Vldlr^{-/-} mice at age of P12. Immunostaining of CD31 at age of 6 wks demonstrated that an injection of the anti-VEGFR2 antibody abrogated the subretinal NV, while the same dose of the anti-VEGFR3 antibody had no effect on subretinal NV, suggesting the role of VEGFR2 in mediating the angiogenic effect of VEGF in this mouse model (FIGS. 2E and 2F).

According to embodiments shown in FIG. 2A, the same amount of eyecup proteins from each mouse was separately blotted with antibodies for VEGF and for VEGFR2. The membranes were stripped and re-blotted with an anti-β-actin antibody. According to embodiments shown in FIG. 2B, VEGF monomer (23 kDa) and dimer (46 kDa) were semi-quantified by densitometry, normalized by β-actin levels and expressed as the relative ratio of wt to Vldlr^{-/-} (mean±SD, n=3). According to embodiments shown in FIG. 2C, the retina was dissected from the eyecup containing the RPE and choroid. Total RNA was separately isolated from these tissues. Real-time RT-PCR showed elevated VEGF mRNA levels in the Vldlr^{-/-} retinas and choroids (expressed as the wt to Vldlr^{-/-} ratio, mean±SD, n=3). According to embodiments and as shown in FIG. 2D, VEGFR2 levels were measured by western blot analysis. According to embodiments and as shown in FIGS. 2E and 2F, purified neutralizing antibodies (rat IgG) for VEGFR2 and VEGFR3 were separately injected into the subretinal space of Vldlr^{-/-} mice at age of P12. The injected antibodies were detected by staining with an FITC-conjugated goat anti-rat antibody at P21 (green). The retinal vasculature was examined using a monoclonal anti-CD 31 antibody (red). Subretinal NV was attenuated by the anti-VEGFR2 antibody but not by the anti-VEGFR3 antibody. The scale of FIGS. 2E and 2F is 10 μm.

### 3. Altered VEGF Subcellular Distribution in RPE Cells in Vldlr^{-/-} Mice

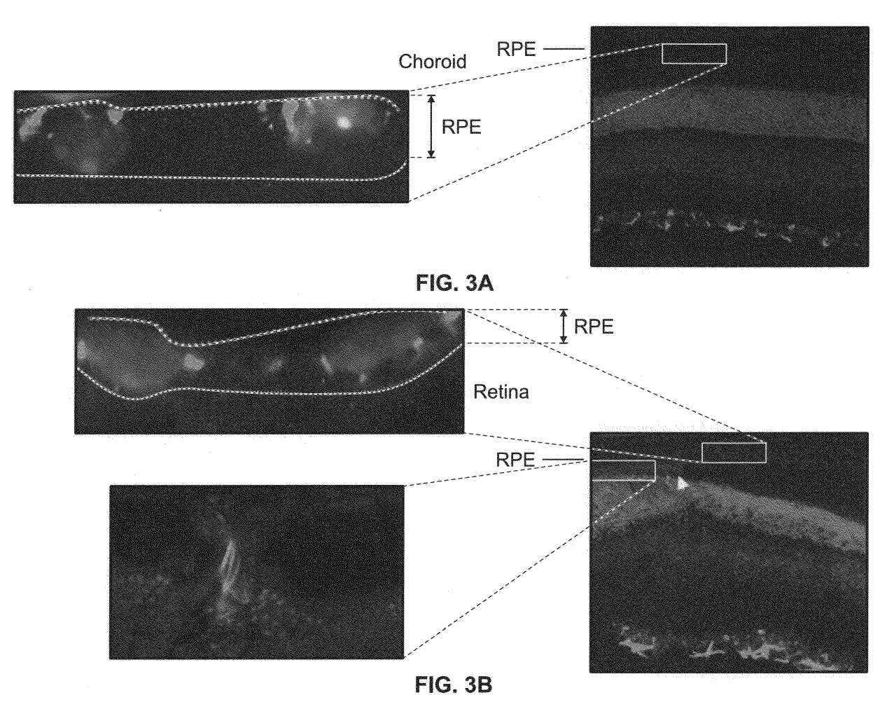

Immunohistochemistry detected intense VEGF signal in the retinal regions displaying NV (FIG. 3). High magnification micrographs showed that VEGF signal in the wt RPE was distributed near the surface adjacent to the Bruch's membrane, but not near the surface adjacent to photoreceptors, consistent with previous observations of the polarized distribution of VEGF in the RPE. In contrast, the VEGF signal in the RPE was detected near the surface adjacent to photoreceptors in some RPE cells in Vldlr^{-/-} mice (FIG. 3).

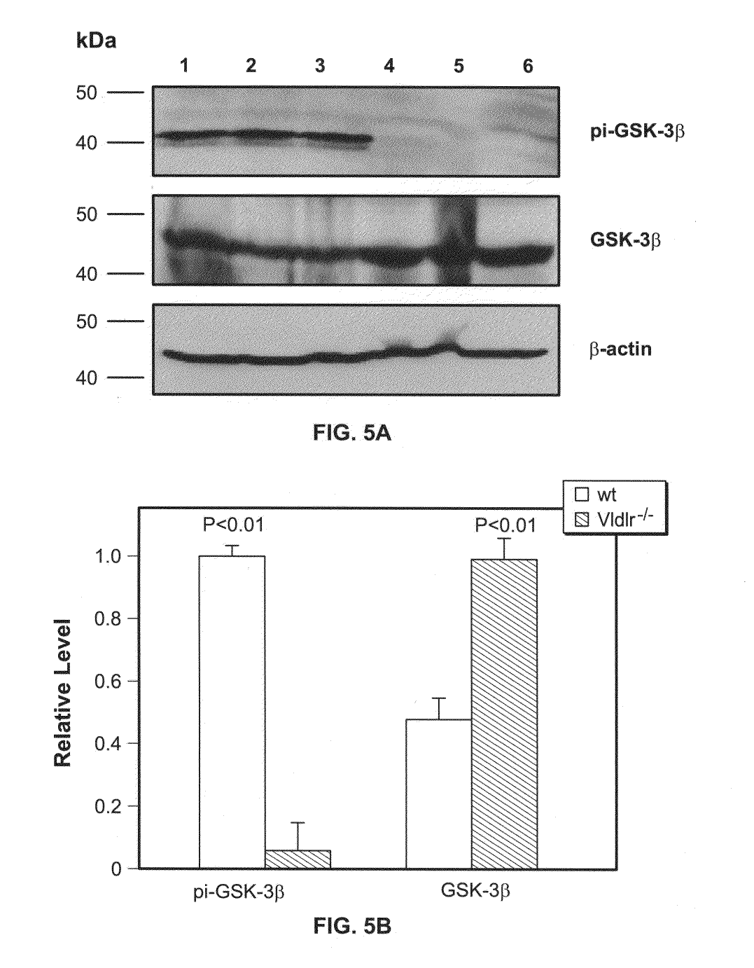

According to embodiments, ocular sections from wt (FIG. 3A) and Vldlr^{-/-} mice (FIG. 3B) were stained with the anti-VEGF antibody (green), and the nuclei counter-stained by DAPI (pseudo-colored red). According to embodiments shown in the magnified box of FIG. 3A showed that VEGF (green, indicated by arrow) is distributed near the RPE surface toward the choroid in wt mice. Similarly, the magnified boxes in FIG. 3B showed that a part of VEGF is distributed near the RPE surface toward the photoreceptors and intensive VEGF signal around the neovascular region. The scale is 20 μm in FIGS. 3A and 3B and 5 μm in the zoomed regions.

### 4. Vldlr Gene Knockout Up-Regulates LRP5/6 Expression in the Evecups

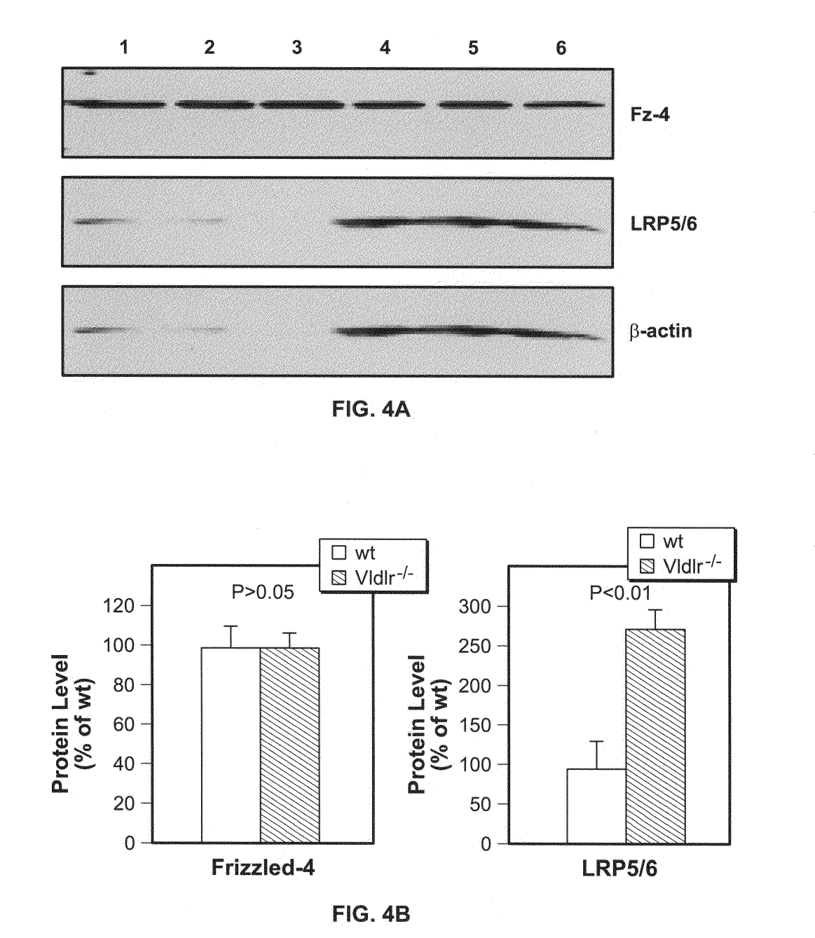

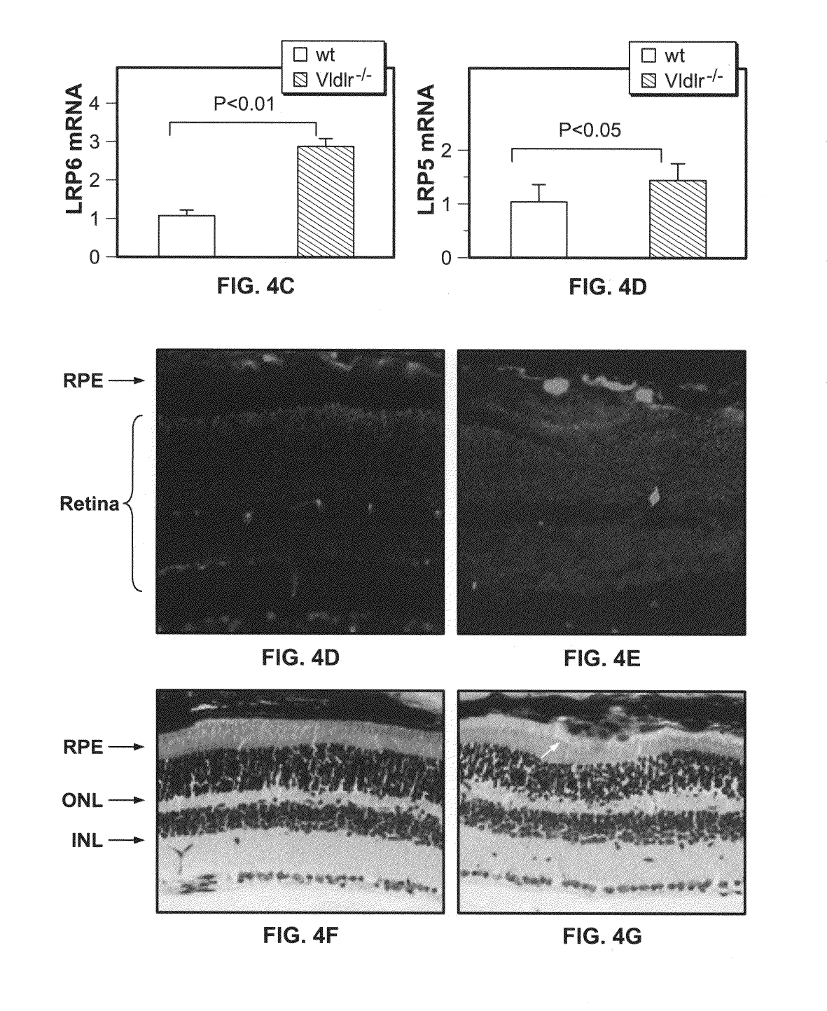

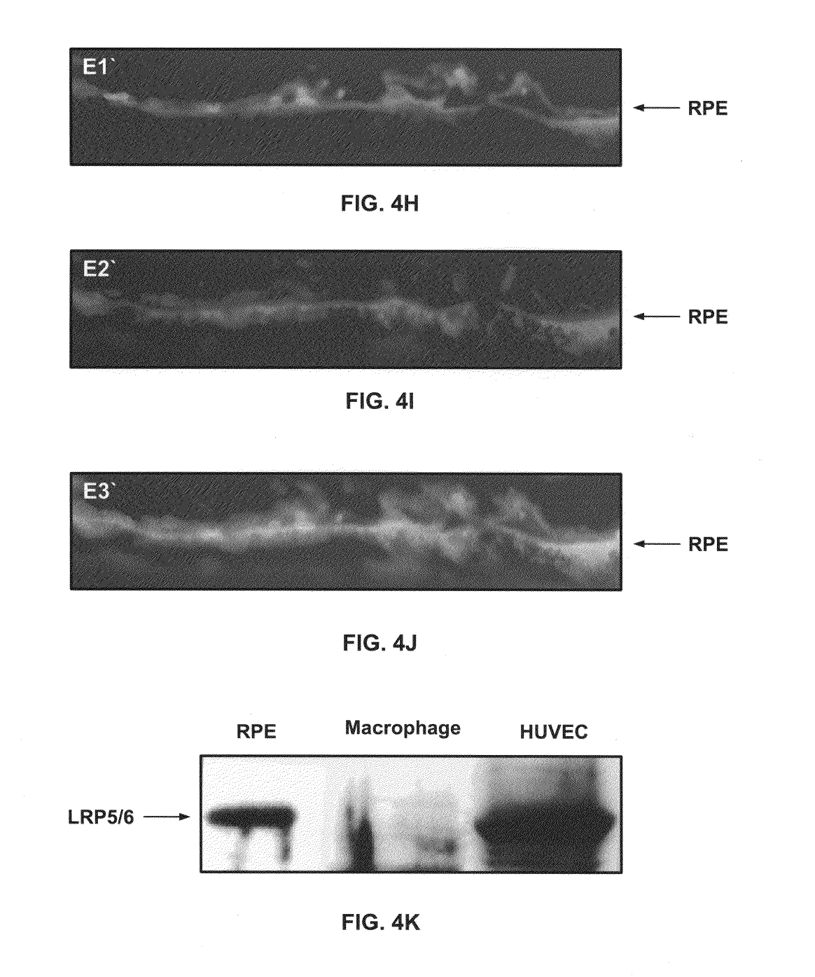

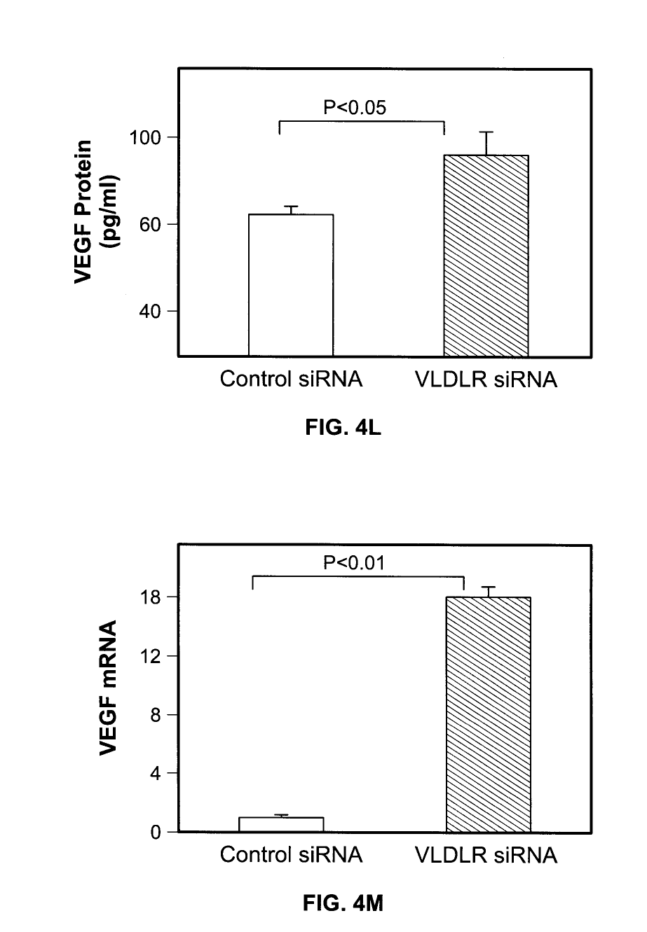

As the wnt signaling pathway is known to play a role in regulation of VEGF expression and in angiogenesis, the expression levels of the wnt receptor Fz4 and the co-receptor LRP5/6 in Vldlr^{-/-} mice with that in wt mice were compared. Western blot and immunohistochemistry analyses showed that LRP5/6 levels were significantly increased in the eyecup of Vldlr^{-/-} mice, when compared to the wt eyecup (FIGS. 4A and 4B). In contrast, Fz4 levels were not changed in the eyecups of Vldlr^{-/-} mice (FIG. 4).

Additionally, to explore whether the loss of VLDLR regulates the expression of LRP5/6 gene expression, LRP5/6 mRNA levels in Vldlr^{-/-} eyecups were compared with that in wt mice using quantitative real-time RT-PCR. The mRNA levels of LRP5 and LRP6 in the Vldlr^{-/-} eyecups increased by 2-fold over that in wt eyecups, indicating that VLDLR gene knockout up-regulates LRP5 and LRP6 gene expression (FIG. 4C).

According to embodiments shown in FIG. 4A, levels of the Fz-LRP5/6 co-receptor were measured by western blot analysis using the anti-Fz4 and anti-LRP5/6 antibodies. In comparison to the wt (lanes 1-3), the Vldlr^{-/-} eyecups (lanes 4-6) did not show apparent differences in Fz4 levels, while showed a significant increase in LRP5/6 levels. According to embodiments shown in FIG. 4B, Fz4 and LRP5/6 levels were semi-quantified by densitometry of western blots, normalized by β-actin levels and expressed as percentages of that in wt (mean±SD, n=3).

According to embodiments shown in FIG. 4C, the LRP5 and LRP6 mRNA were quantified in Vldlr^{-/-} and wt eyecups using quantitative real-time RT-PCR. The relative mRNA levels are expressed as fold over the wt control (mean±SD, n=3). According to embodiments shown in FIGS. 4D and 4E, immunohistochemistry of LRP5/6 in the eye sections of wt (FIG. 4D) and Vldlr^{-/-} mice (FIG. 4E), showing an increased LRP5/6 signals in the subretinal space of Vldlr^{-/-} mice. 200×. In FIGS. 4H-4J, double immunostaining of LRP5/6 (FIG. 4H) and RDH10, an RPE marker (FIG. 4I) in the Vldlr^{-/-} mouse eyecup showed that there is a co-localization of RDH10 (red) and LRP5/6 (green) signals in the RPE. FIG. 4J is merged image of FIG. 4E1′ and FIG. 4E2′. 630×. According to embodiments shown in FIGS. 4F and 4G, H&E staining of the ocular sections from wt (FIG. 4F) and Vldlr^{-/-} mice (FIG. 4G).

According to embodiments shown in FIG. 4K, the same amount of cell lysates from bovine RPE, human mono/macrophages, and HUVEC cells were blotted with the anti-LRP5/6 antibody which identified a band with expected molecular weight in HUVEC and RPE cells but not in macrophages.

According to embodiments shown in FIGS. 4I and 4J, regulation of VEGF expression by VLDLR is shown. HUVEC cells were transfected with the VLDLR siRNA or the control siRNA. VEGF expression was quantified by ELISA using the conditioned medium (I) and by real-time RT-PCR at the mRNA level in FIG. 4J (mean±SD, n=3).

### 5. Vldlr Gene Knockdown Up-Regulates VEGF Expression in Endothelial Cells and RPE Cells

To identify the cell types in which the regulation of LRP5/6 by VLDLR occurs, double immunostaining of LRP5/6 and an RPE marker (RDH10) in the Vldlr^{-/-} mouse eyecup was performed. The results showed a co-localization of RDH10 and LRP5/6 in the RPE (FIGS. 4H-4J).

The expression of LRP5/6 in primary RPE cells from bovine eyes, HUVEC, and in human mono/macrophages was examined. The equal amount of proteins from these lysates was blotted with an anti-LRP5/6 antibody. LRP5/6 was detected in the RPE cells and HUVEC, with the expected molecular weight as that in HUVEC, but not in the macrophages (FIG. 4K).

In order to reveal if VLDLR regulates VEGF expression at the protein or mRNA level, HUVEC cells were transfected with the VLDLR siRNA, using an siRNA with scrambled sequence as negative control. The culture medium was collected and concentrated 48 hours after the transfection for VEGF ELISA. VEGF secreted from both the HUVEC cells transfected with the VLDLR siRNA were significantly higher than in the cells transfected with the control siRNA (FIG. 4L). The VEGF mRNA levels were also significantly elevated by the VLDLR siRNA (FIG. 4M)

### 6. Activation of Down-Stream Effectors of the Wnt Signaling Pathway in Vldlr^{-/-} Eyecups

The activation status of GSK-3β and β-catenin, two down-stream effectors of the wnt signaling pathway, was determined by measuring their phosphorylation status in Vldlr^{-/-} mouse eyecups. The levels of phosphorylated and free GSK-3β in Vldlr^{-/-} eyecups were compared with that in wt by western blot analysis using specific antibodies. Vldlr^{-/-} mouse eyecups showed dramatically decreased levels of phosphorylated GSK-3β but elevated free GSK-3β in the eyecups, when compared to that in wt mice.

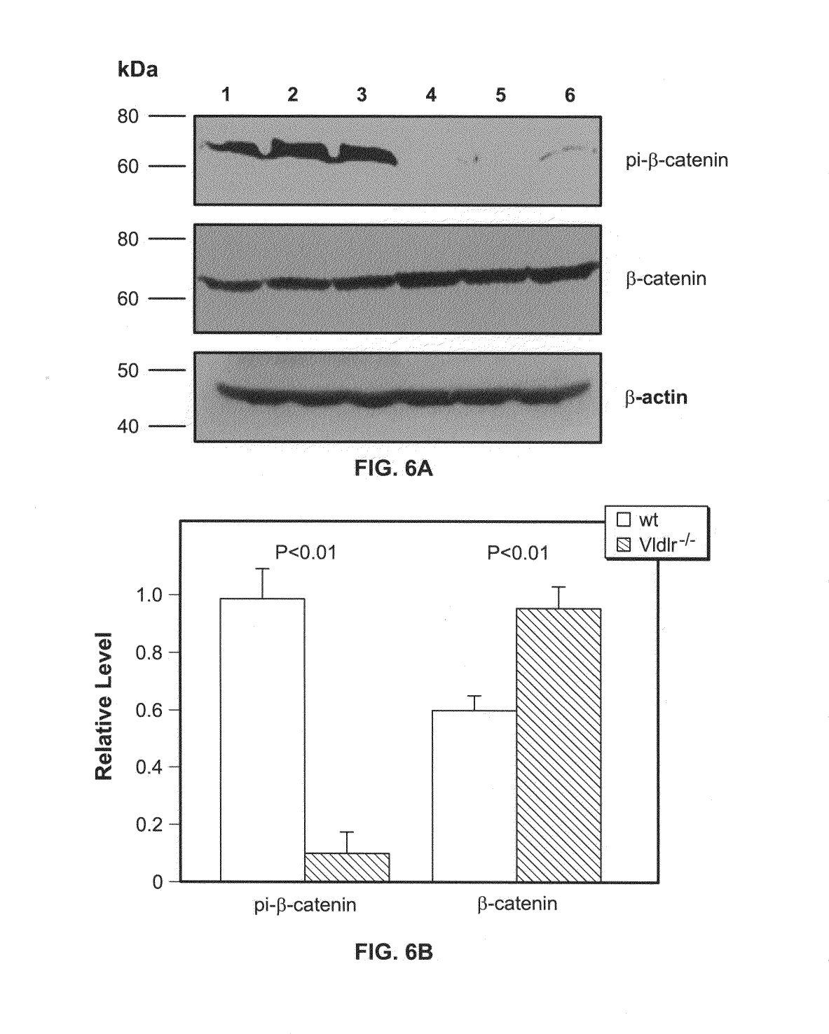

According to embodiments of experimental data and shown in FIG. 5A, the same amount of eyecup proteins (100 μg) from each mouse was blotted separately with antibodies for phosphorylated GSK-3β and for free GSK-3β. The phosphorylated GSK-3β levels in Vldlr^{-/-} eyecups (lanes 4-6) were drastically decreased compared to those in wt mice (lanes 1-3). Conversely, levels of free GSK-3β in Vldlr^{-/-} eyecups (lanes 4-6) were apparently higher than in wt eyecups (lanes 1-3). According to embodiments shown in FIG. 6B, phosphorylated and free GSK-3β were semi-quantified by densitometry, normalized by β-actin levels and expressed as relative ratios between the wt and Vldlr^{-/-} eyecups (mean±SD, n=3).

Similar to what was observed with GSK-3β, phosphorylation of β-catenin, a downstream target of GSK-3β, was nearly completely abolished in Vldlr^{-/-} mouse eyecups as shown by western blot analysis using an antibody specific for phosphorylated β-catenin (FIG. 6). The free form of β-catenin was increased in Vldlr^{-/-} mice when compared to that in wt mice, demonstrating an accumulation of β-catenin in Vldlr^{-/-} eyecups (FIG. 6).

According to embodiments shown in FIG. 6A, the same amount of eyecup proteins (100 μg) was blotted separately with antibodies for phosphorylated β-catenin and for free β-catenin. Levels of phosphorylated β-catenin were undetectable in Vldlr^{-/-} eyecups (lanes 4-6), while intensive signal was detected in wt mice (lanes 1-3). In contrast, levels of free β-catenin in Vldlr^{-/-} eyecups (lanes 4-6) were higher than that in wt eyecups (lanes 1-3). According to embodiments shown in FIG. 6B, phosphorylated and free β-catenin were semi-quantified by densitometry and normalized by β-actin levels (mean±SD, n=3).

### 7. VLDLR Regulates the Wnt Signaling in Endothelial Cells

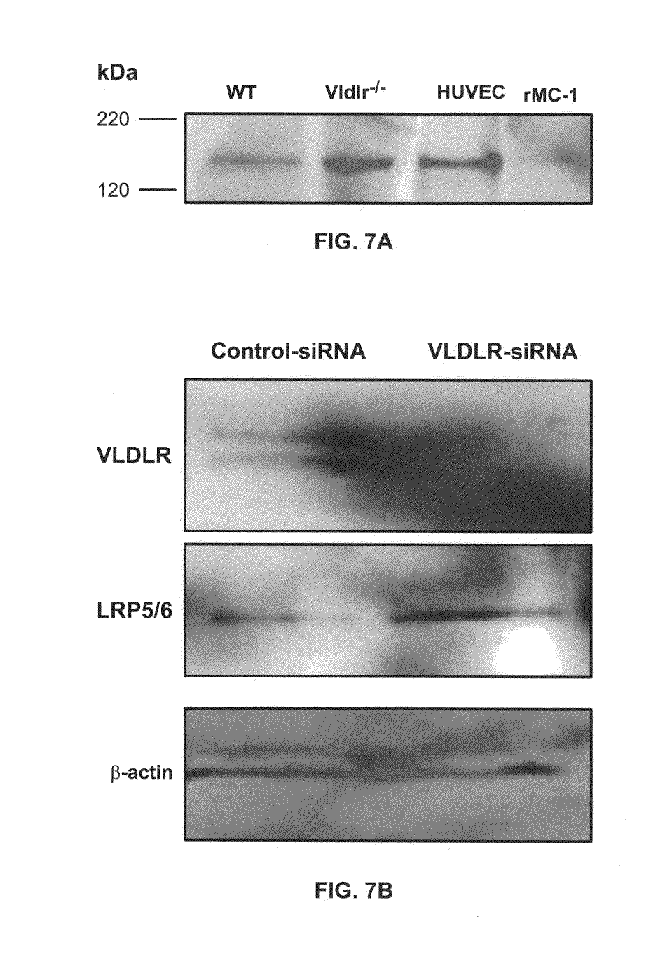

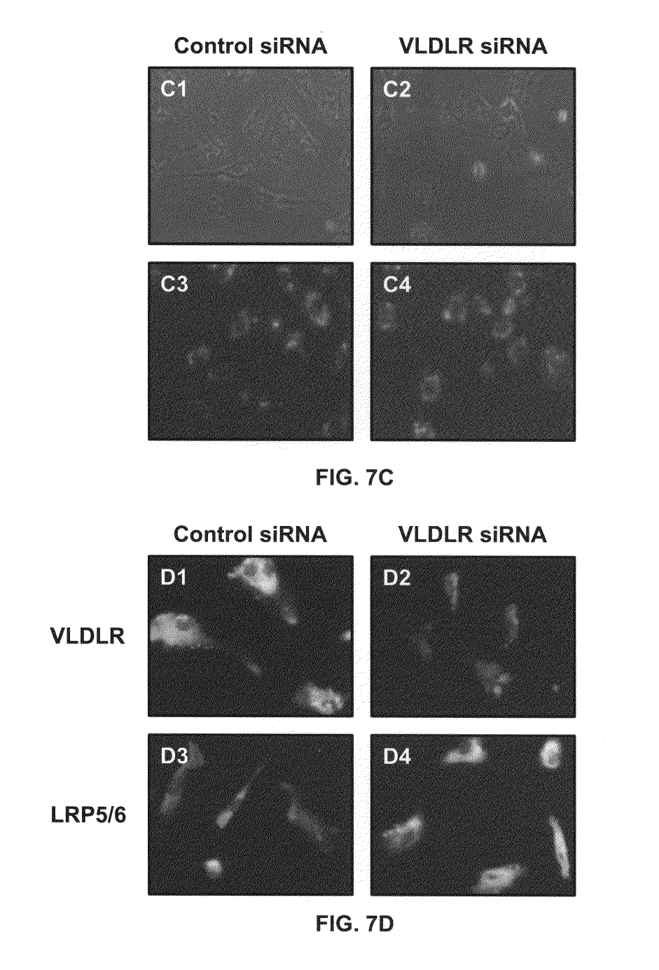

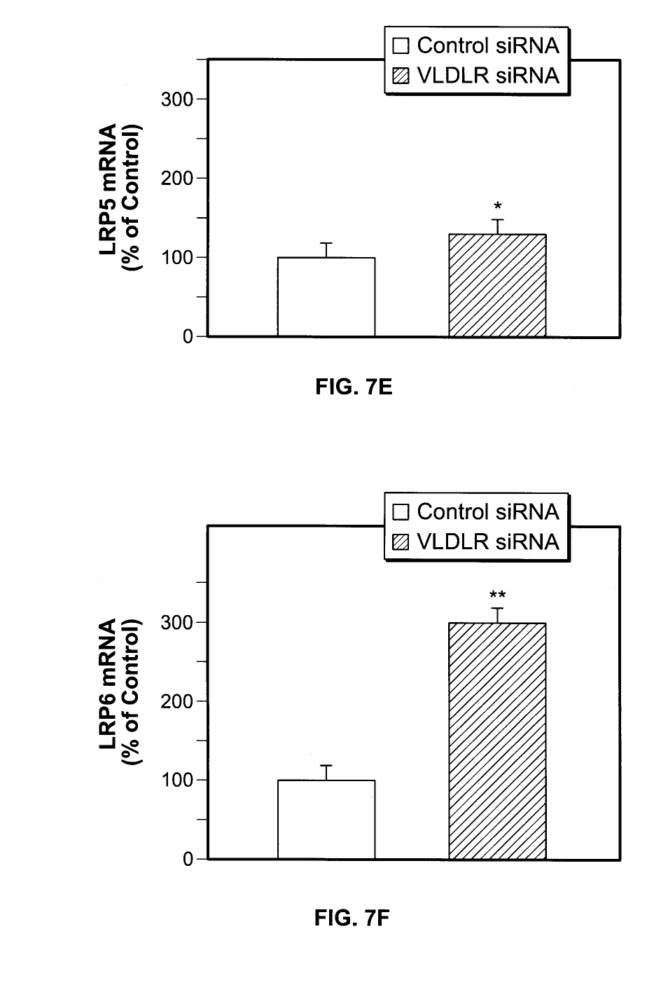

To further confirm that VLDLR directly regulates the wnt signaling, VLDLR and LRP5/6 were determined to be located in the same cells. As VLDLR is well known to express at high levels in endothelial cells, the expression of LRP5/6 in HUVEC was determined. Western blot analysis showed that LRP5/6 is expressed at high levels in HUVEC with the same molecular weight as that in the mouse eyecups, but not in rMC-1, a rat Müller cell line (FIG. 7A).

To establish the regulatory function of VLDLR in the activation of the wnt signaling pathway, siRNA was used to knock down the expression of VLDLR in HUVEC and measured the expression levels of LRP5/6 and subcellular localization of β-catenin, which is a down-stream effector of the wnt signaling pathway and directly up-regulates VEGF expression in endothelial cells. As shown by western blot analysis and immunocytochemistry, the siRNA specific for VLDLR decreased VLDLR levels in HUVEC. In contrast, the siRNA significantly up-regulated LRP5/6 expression in the same cells, compared to the cells treated with the same amount of the control siRNA with a scrambled sequence (FIGS. 7B-7D). Real-time RT-PCR also showed that the VLDLR siRNA up-regulated LRP5 and LRP6 mRNA levels (FIG. 7E-7F).

According to embodiments of experimental data, FIG. 7A demonstrates high levels of LRP5/6 in endothelial cells. Total proteins from wt and Vldlr^{-/-} eyecups, cultured HUVEC and rMC-1 cells were immunoblotted with an antibody specific for LRP5/6. According to embodiments of experimental data shown in FIG. 7B, down-regulation of VLDLR by a specific siRNA is demonstrated. HUVEC were transfected with the Cy3-labeled VLDLR siRNA or control siRNA. Twenty-four hours after the transfection, 15 μg of total cellular proteins were separately blotted with an anti-VLDLR antibody and anti-LRP5/6 antibody. According to embodiments of experimental data shown in FIG. 7C, HUVEC were transfected with the Cy3-labeled VLDLR siRNA (C2, C4) and control siRNA (C1, C3) to show high transfection efficiency. (C1) and (C2) are phase contrast images of (C3) and (C4), respectively. According to embodiments shown in FIG. 7D, the transfected cells were separately stained with the anti-VLDLR antibody (green) (D1, D2) and with an anti-LRP5/6 antibody (green) (D3, D4). Red color, Cy3 signaling from the siRNAs. The scale for the slices in FIGS. 7C-7D is 10 μm. Note that LRPE5/6 signal was increased by VLDLR siRNA (D4). According to embodiments shown in FIG. 7E, the mRNA of LRP5 (E1) and LRP6 (E2) was quantified by real-time RT-PCR and compared using Student's t test. Values are the relative LRPE5 and LRPE6 mRNA leveles as fold over that in the cells treated with the control siRNA (mean±SD, n=3). *P<0.05; **P<0.01.

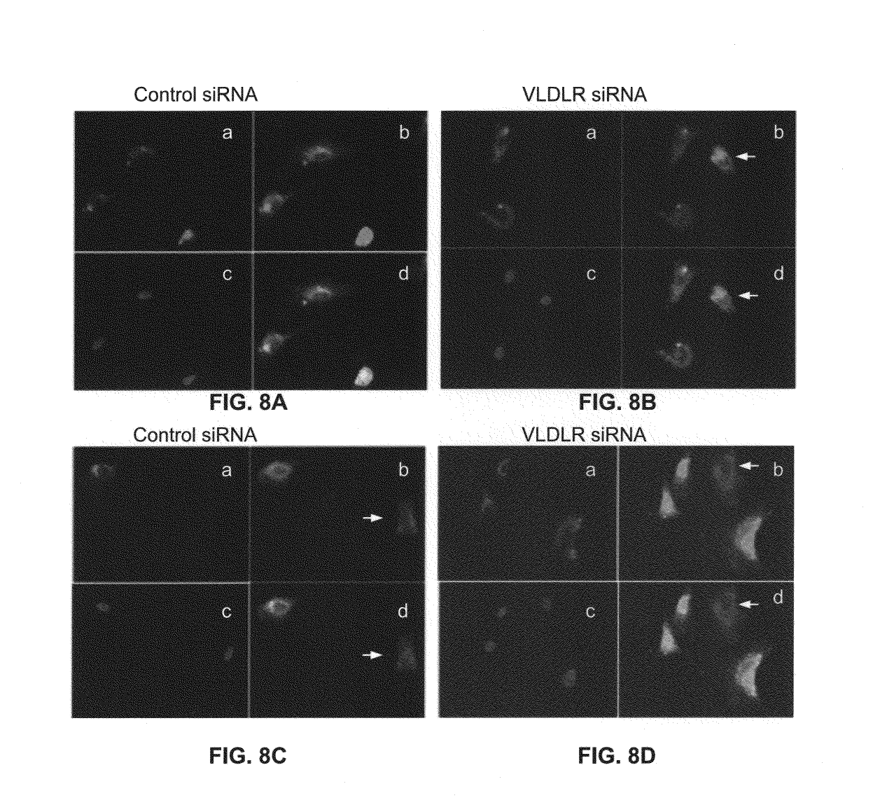

Immunocytochemistry using the antibody specific for phosphorylated β-catenin showed that the treatment of HUVEC with the VLDLR siRNA reduced phosphorylation of β-catenin (FIGS. 8A-8B). Immunostaining with an antibody specific for free β-catenin demonstrated increased levels of free β-catenin in cells treated with the VLDLR siRNA, compared to that treated with the control siRNA. Moreover, the VLDLR siRNA treatment induced an increased nuclear translocation of β-catenin, a key step in its activation (FIGS. 8C-8D). These results indicate that down-regulation of VLDLR alone activates the wnt signaling pathway in retinal endothelial cells.

HUVEC were separately transfected with the Cy3-labeled VLDLR siRNA and control siRNA (red). Twenty-four hours following the transfection, the cells were immunostained with an antibody specific for phosphorylated β-catenin (embodiments in FIGS. 8A and 8B), and with antibody specific for free β-catenin (green)(embodiments in FIGS. 8C and 8D). The nucleus was counter-stained with DAPI (blue). (a) Cy3 signal; (b) β-catenin staining; (c) DAPI staining; (d) merged image of (a, b, c). Arrows indicate an untransfected cell with lower nuclear levels of β-catenin. The scale for FIG. 8 is 10 μm.

### 8. Inhibition of VEGF Expression by a Specific Inhibitor of the Wnt Signaling Pathway in Vldlr^{-/-} Mice

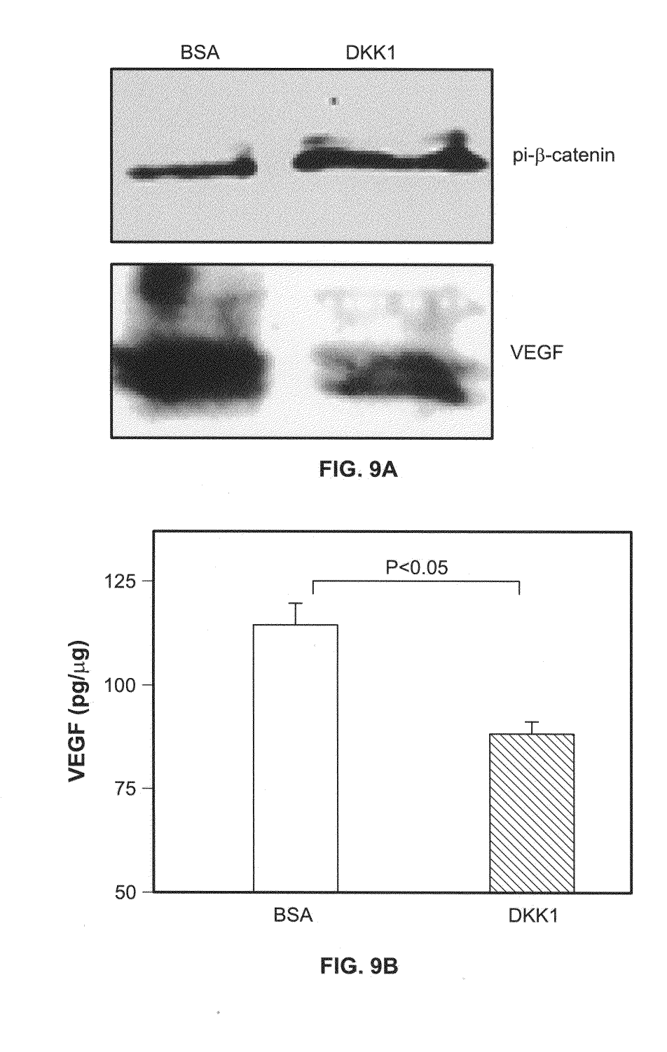

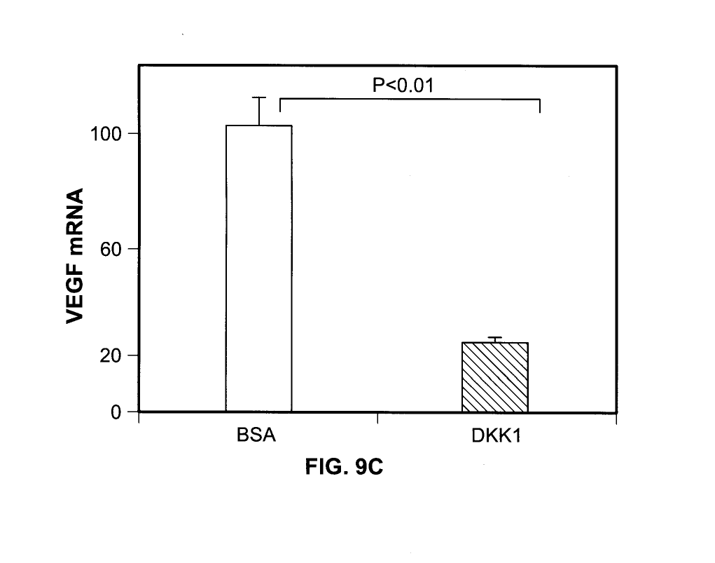

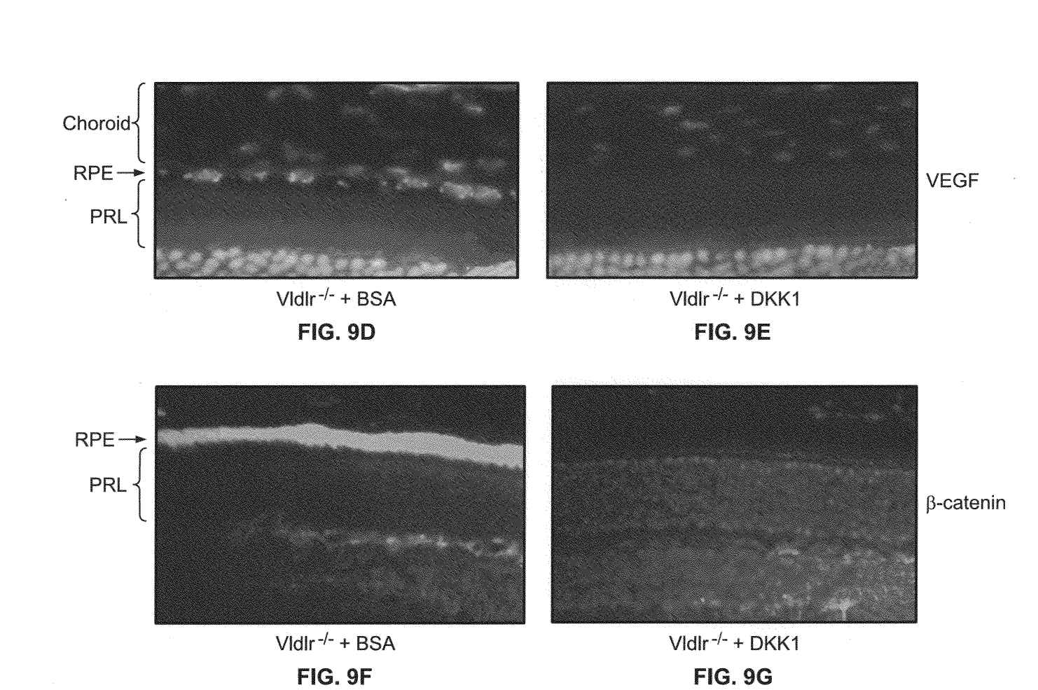

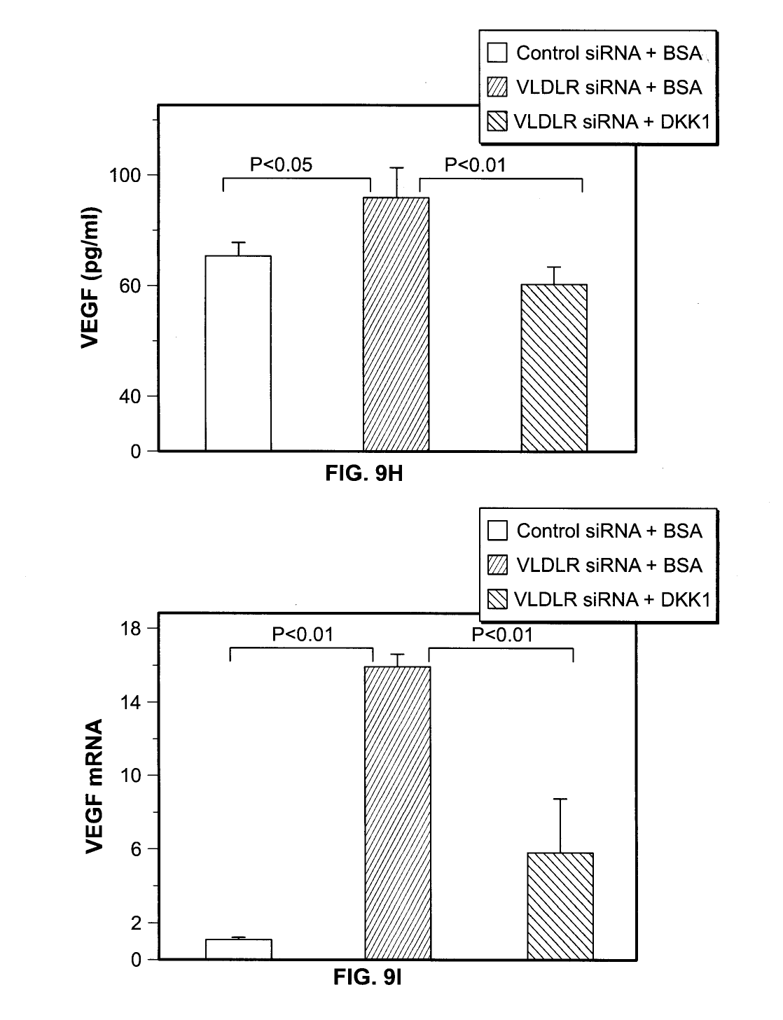

To confirm the association of the wnt pathway activation with VEGF over-expression in Vldlr^{-/-} mice, 5 μg purified mouse DKK1, a specific inhibitor of the wnt signal pathway by binding with LRP5/6, were injected into the subretinal space of the Vldlr^{-/-} mice into one eye and the same amount of a control protein (BSA) into the contralateral eye. Twenty-four hours after the injection, β-catenin phosphorylation and VEGF levels in the eyecup were measured by western blot analysis. The results showed that DKK1 increased phosphorylation of β-catenin, suggesting an inhibited wnt signaling pathway (FIG. 9A). As shown by western blot analysis and real-time RT-PCR, DKK1 also significantly decreased the VEGF protein and mRNA levels in Vldlr^{-/-} eyecups, correlating with the inhibited wnt signaling (FIGS. 9B and 9C). Immunohistochemistry using antibodies specific for VEGF and β-catenin showed that a sub-retinal injection of DKK1 significantly decreased levels of VEGF and β-catenin in the RPE of Vldlr^{-/-} mice, compared to the contralateral eyes injected with the same amount of BSA (FIGS. 9D-9G).

The regulatory roles of VLDLR and the wnt pathway in VEGF expression in cultured endothelial cells was also investigated. In HUVEC cells, DKK1 also blocked the VEGF over-expression induced by the VLDLR siRNA (FIGS. 9H and 9I). Consistent with documented evidence, these new data suggest that the activated wnt signaling pathway is responsible, at least in part, for the VEGF over-expression in Vldlr^{-/-} mice.

Vldlr^{-/-} mice received a subretinal injection of 5 μg purified mouse DKK1 into the right eyes, and the same amount BSA into the left eyes. According to embodiments shown in FIG. 9A, The eyecups were dissected 24 h following the injection for the following analyses, phosphorylated β-catenin and VEGF levels were measured by Western blot analysis of a representative mouse eyecup using the anti-phosphorylated β-catenin and anti-VEGF antibodies, respectively. According to embodiments shown in FIG. 9B, VEGF protein levels in Vldlr^{-/-} eyecups were measured using ELISA. According to embodiments shown in FIG. 9C, VEGF mRNA levels in the Vldlr^{-/-} eyecups were measured using quantitative real-time RT-PCR. Values are mean±SD (n=4). According to embodiments shown in FIGS. 9D-9G, Vldlr^{-/-} mice were injected with 5 μg/eye BSA into the subretinal space of the left eye (FIGS. 9D and 9F) and the same amount of DKK1 into the right eye (FIGS. 9E and 9G). The ocular sections were immunostained with an anti-VEGF antibody (FIGS. 9D and 9E) and an anti-β-catenin antibody (FIGS. 9F and 9G). 400×. PRL, photoreceptor layer. According to embodiments shown in FIGS. 9H and 9I, HUVEC were transfected with the VLDLR siRNA. The transfected cells were treated with 10 μg/ml human DKK1or BSA. VEGF protein was measured in the culture medium by ELISA (FIG. 9H) and the VEGF mRNA measured in the cells by real-time RT-PCR, normalized by 18s RNA and expressed as arbitrary unit (mean±SD, n=3) (FIG. 9I), 24 hours after the addition of DKK1.

### 9. The Wnt Pathway is Activated in the Retina from Human Patients with Diabetic Retinopathy

Retinal levels of total β-catenin, a down-stream effector of the wnt pathway, was examined in the retinal sections from two non-diabetic human donors (non-DM) and two diabetic donors with non-proliferative retinopathy (DM-NPDR) using immunohistochemistry. Retinal sections from patients with DM-NPDR showed more intensive β-catenin staining compared to the non-DM patients. Moreover, elevated β-catenin levels in the nucleus of inner retinal cells were observed in the DM-NPDR patients, confirming the wnt pathway is activated in the human retina with diabetic retinopathy.

According to embodiments of experimental data, retinal sections from two diabetic patients with NPDR (FIGS. 10C and 10D) and two non-diabetic (non-DM) subjects (FIGS. 10A and 10B) were immunostained with an antibody for β-catenin, and the signal developed with the DAB method (brown color). Note the more intensive signal of β-catenin in the inner retina and increased staining in the nuclei of the retinal cells in the DM-NPDR patients, compared to that from the non-DM subjects. The scale for FIGS. 10A-10D is 20 μM where the bar is shown.

### 10. Activation of the Wnt Pathway in the Retina of Akita Mice

Akita mice were examined for the activation status of the wnt pathway in retina. The retinas were dissected and homogenized from three male Akita mice with confirmed hyperglycemia and three wt mice at age of 16 weeks. Western blot analysis showed apparently elevated β-catenin levels in Akita mouse retinas, compared to the age-matched wt mice with the same genetic background. These data suggest that similar to STZ-induced diabetic rats and oxygen-induced retinopathy rats, the wnt pathway is activated in the retina of this genetic diabetic model as well.

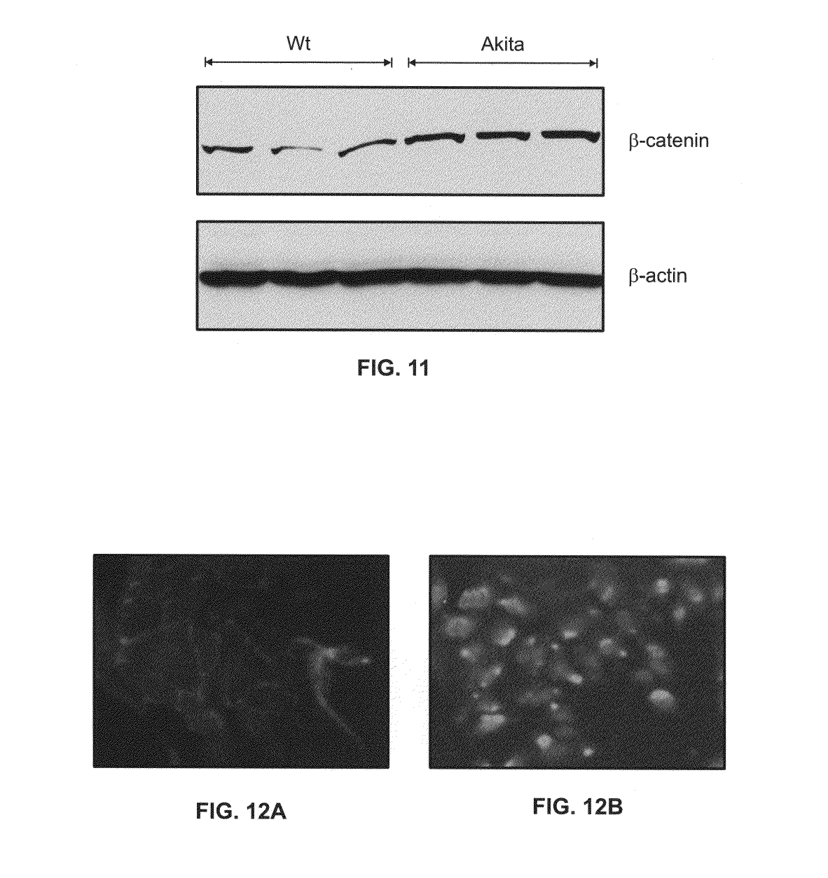

According to embodiments of experimental data and as shown in the western blot of FIG. 11, the retinas of three Akita mice at age 16 weeks and age-matched wt controls were dissected and homogenized. The same amount of retinal proteins from each animal was blotted with an antibody for β-catenin and normalized using β-actin. Each lane shown in FIG. 11 represents an individual animal.

### 11. Oxidative Stress is Responsible for the Wnt Pathway Activation Induced by High Glucose

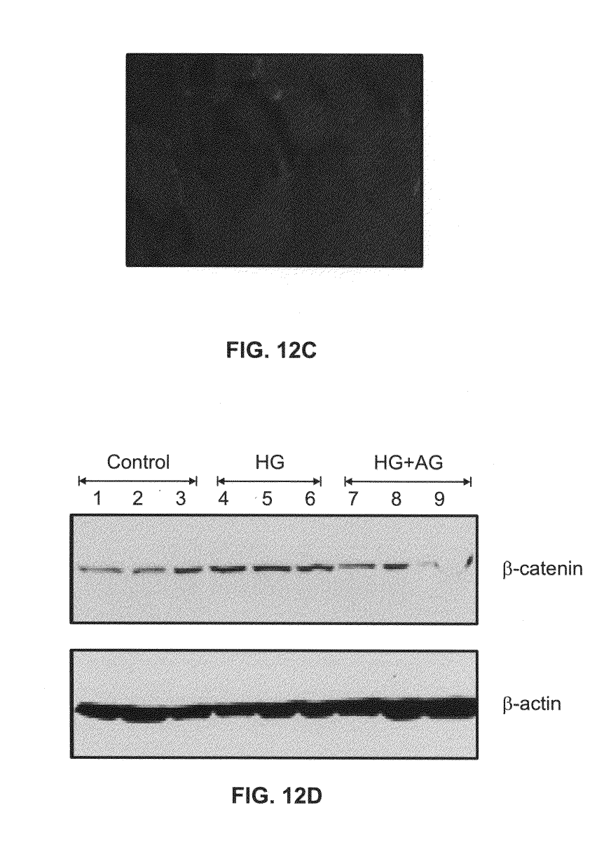

In preliminary studies presented in the proposal, high glucose has been shown to be a causative factor for the activation of the wnt signaling in diabetes. To elucidate the mechanism for of the wnt pathway activation induced by high glucose, primary bovine retinal capillary endothelial cells (BRCEC) were treated with low glucose (5 mM glucose and 20 mM mannitol), high glucose (30 mM glucose), and high glucose together with a known anti-oxidant (30 mM glucose with 10 μM aminoguanidine) for 24 h. The nuclear levels of β-catenin were examined by immunocytochemistry using the antibody for β-catenin. The results showed that high glucose induced a significant increase of nuclear levels of β-catenin (see FIG. 12). The nuclear translocation of β-catenin induced by high glucose was blocked by aminoguanidine. Consistent with this immunohistochemistry results, western blot analysis showed that total levels of β-catenin in cultured BRCEC were increased by high glucose, but blocked by the anti-oxidant. These results suggest that oxidative stress induced by high glucose levels is responsible, at least partially, for the wnt pathway activation in diabetes.

According to embodiments of experimental data, cultured BRCEC were exposed to 5 mM glucose plus 20 mM mannitol as control (FIG. 12A), 30 mM glucose (FIG. 12B), and 30 mM glucose plus 10 μM aminoguanidine (AG) (FIG. 12C) for 24 hours. The sub-cellular distribution of β-catenin was revealed by immunocytochemistry using the antibody for β-catenin. In FIG. 12D, protein levels of β-catenin were determined using western blot analysis, and normalized by β-actin levels. Each lane of FIG. 12D represents an individual culture dish.

### 12. DKK1 Inhibits Inflammation in the Retina of Diabetic Rats

To determine the role of wnt pathway in retinal inflammation in diabetic retinopathy, different doses of purified DKK1, a specific inhibitor of the wnt pathway, was injected into the vitreous of the right eye of STZ-diabetic rats. As shown by ELISA, diabetes induced over-expression of ICAM-1 in the retina. DKK1 inhibited the ICAM-1 over-expression in a dose-dependent manner, compared to the contralateral eyes injected with the same amount of BSA, indicating that the wnt signaling activation in diabetes contributes to retinal inflammation in diabetes.

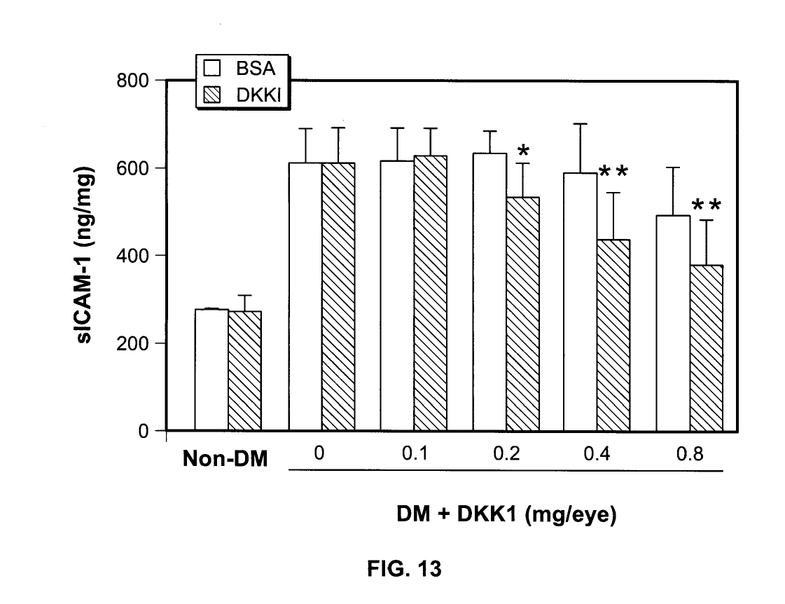

According to embodiments of experimental data shown in FIG. 13, different doses of purified DKK1 were injected intravitrealy into the right eye of STZ-diabetic rats at 16 weeks following the onset of diabetes, and the same amounts of BSA into the left eye for control. Soluble ICAM-1 was measured at 2 days after the injection using the sICAM-1 ELISA kit, normalized by total protein concentrations and expressed as ng.

### 13. DKK1 Attenuates ROS Generation Induced by High Glucose

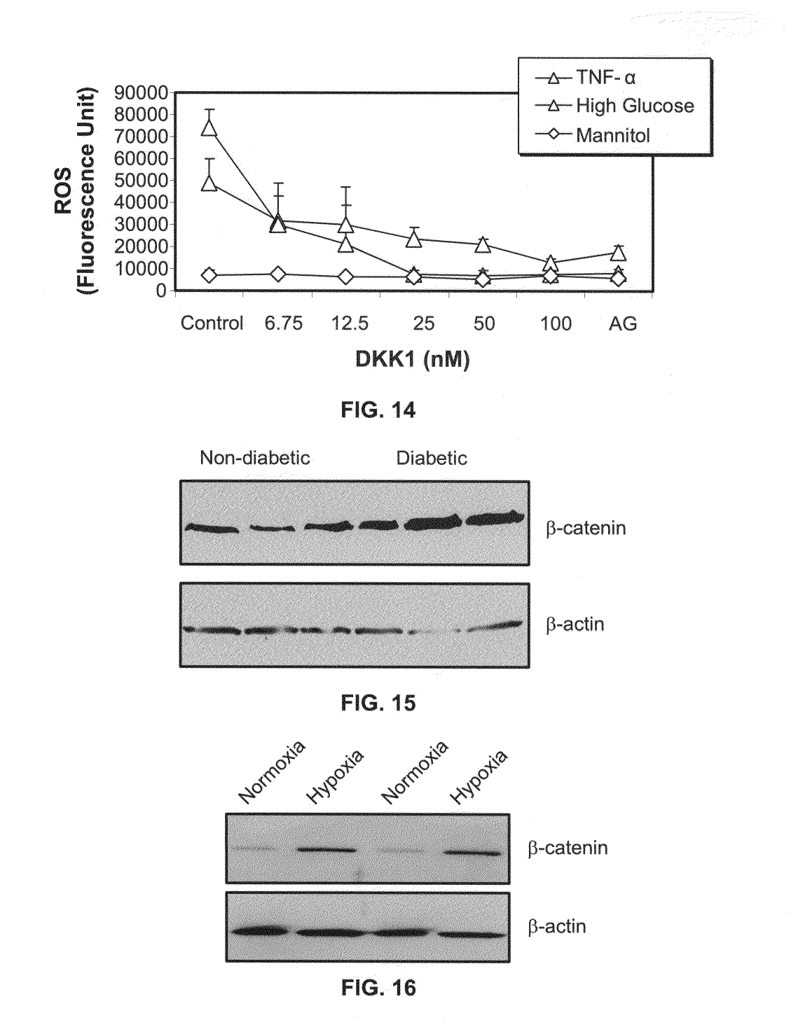

A primary culture of bovine retinal capillary endothelial cells (BRCEC) were exposed to 30 mM glucose or TNF-α in the absence or presence of various concentrations of DKK1 (6.25-100 nM) for 2 h. Aminoguanidine was used as a positive control as it is a known inhibitor of ROS generation. As shown by the intracellular ROS generation assay, DKK1 blocked the ROS generation induced by high glucose and TNF-α (FIG. 14), suggesting that the wnt pathway is mediating the ROS generation in diabetes. These results provide further support for our hypothesis that the wnt pathway activation plays a pathogenic role in diabetic retinopathy.

According to embodiments of experimental data shown in FIG. 14, BRCEC were exposed to 30 mM glucose or 1 μg/ml TNF-α in the absence or presence of various concentrations of DKK1 (6.25-100 nM) for 2 h, 10 μM aminoguanidine (AG) was used as positive control. Intracellular ROS generation was measured using CM-H₂DCFDA, and expressed as fluorescent unit per well (means±SD, n=3).

### 14. β-Catenin is Accumulated in the Retina of STZ-Induced Diabetic Rats

To determine if the wnt pathway is activated by diabetes, retinal levels of β-catenin, the key effector of the wnt signaling pathway, were measured by western blot analysis in BN rats with STZ-induced diabetes and compared to age-matched non-diabetic controls. The STZ-diabetic rats showed significantly elevated retinal β-catenin levels in the retina, compared to those in non-diabetic controls of the same age.

According to embodiments of experimental data and as illustrated in FIG. 15, the retinas of diabetic BN rats were dissected at 16 weeks following the STZ injection and age-matched non-diabetic normal control. The same amount of soluble proteins from each rat were blotted with an antibody specific for β-catenin and normalized by β-actin levels. Each lane represents an individual rat.

### 15. Hypoxia Up-Regulates β-Catenin Expression in Cultured Retinal Capillary Endothelial Cells (RCEC)

To identify the causative factor responsible for the wnt pathway activation in the OIR and diabetic retina, primary RCEC were exposed to hypoxia (1% oxygen) for 3 days. The results showed that the hypoxia exposure significantly up-regulated β-catenin levels (FIG. 16), suggesting that the wnt pathway is activated by hypoxia in RCEC.

According to embodiments and as shown in FIG. 16, primary bovine RCEC were cultured in an EC culture medium under normoxia and hypoxia (1% oxygen) for 3 days. The same amount of total protein from each dish was blotted with an antibody for β-catenin and reblotted with an antibody for β-actin. The blot represents two independent experiments side by side.

### 16. DKK1, a Specific Inhibitor of the Wnt Pathway. Inhibits HIF-1 Activation and VEGF Expression Induced by the Wnt Signaling

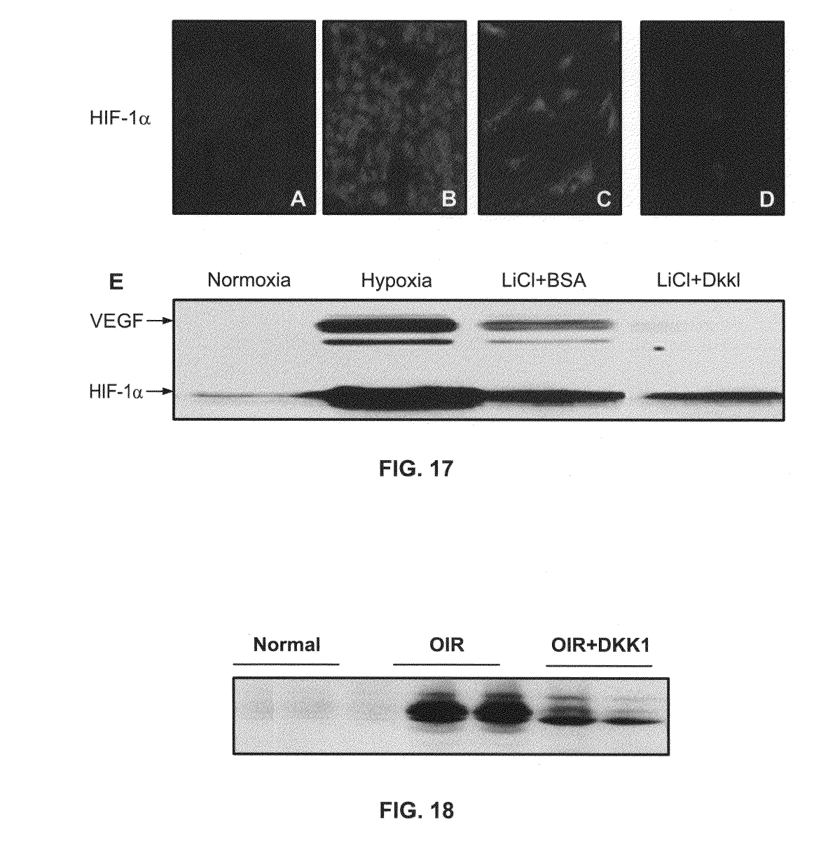

Blockade of the wnt pathway by DKK1 down-regulates VEGF and HIF-1 expression and reduces vascular leakage in the retina of diabetic rats. To activate the wnt pathway, primary RCEC were treated with 50 mM LiCl, a specific activator of the wnt pathway, in the presence or absence of 85 ng/ml DKK1, a specific inhibitor of the wnt pathway, or BSA for 24 hours. Hypoxia was used as a positive control for HIF-1 activation. The HIF-1 nuclear translocation, a key step in its activation, was measured by immunocytochemistry. Both LiCl and hypoxia increased nuclear levels of HIF-1α, indicating an increased nuclear translocation. DKK1 inhibited the HIF-1 nuclear translocation induced by LiCl (FIGS. 17A-17D).

According to embodiments of experimental data shown in FIG. 17E and as measured by western blot analysis, hypoxia and LiCl both up-regulated the expression of HIF-1α and VEGF. DKK1 blocked the HIF-1α and VEGF over-expression induced by the activation of the wnt pathway, suggesting an anti-angiogenic activity of this wnt pathway inhibitor.

According to embodiments of experimental data, RCEC were exposed to normoxia (FIG. 17A), hypoxia (1% O₂) (FIG. 17B), 50 mM LiCl with BSA (FIG. 17C), or 50 mM LiCl plus 85 ng/ml DKK1 (FIG. 17D) for 24 hours. The nuclear translocation of HIF-1α was determined by immunocytochemistry with an antibody against HIF-1α. Note hypoxia and LiCl both significantly increased HIF-1α levels in the nucleus, which was blocked by DKK1. In FIG. 17E, the total levels of VEGF and HIF-1α were determined by western blot analysis with an antibody against HIF-1α or VEGF.

### 17. DKK1 Blocks the VEGF Over-Expression in the Retina of OIR Rats

To determine the effect of the wnt pathway blockade of the VEGF expression induced by ischemia, DKK1 was injected into the vitreous of the OIR rats at P15. The rats were then maintained in normoxia for 3 days, and the VEGF expression in the retina was measured. As shown by western blot analysis, VEGF levels in the retina were dramatically increased in the OIR rats injected with BSA, compared to normal rat retina at the same age. Injection of 3 μl of 1 μg/μl DKK1 significantly blocked VEGF over-expression, compared to OIR control eyes which received the same amount of BSA. These results suggest that a single DKK1 injection partially decreases the expression of VEGF in the retina of the OIR model.

According to embodiments of experimental data, and as illustrated in FIG. 18, DKK1 was injected into the vitreous cavity of OIR rats (3 μg/eye) at P15, and the retinas were harvested at P18. The same amount of BSA was injected as control. The same amount of total protein from each rat was used for western blot analysis using specific antibody for VEGF. Each lane represents an individual rat.

### 18. DKK1 Mitigates Retinal NV in the OIR Model

To determine the anti-angiogenic effect of DKK1 on retinal NV, DKK1 was injected (3 μl of 1 μg/μl DKK1) into the vitreous of the OIR rats at P15. At the time, NV had partially formed in the OIR rats at P15. The rats were then maintained in normoxia for 3 days, and then the retinal NV was examined using fluorescein angiography on retinal whole mounts at P18. Eyes injected with DKK1 showed decreased retinal NV areas, compared to the control eyes injected with the same amount of BSA. These results suggest that a single DKK1 injection partially blocks the retinal NV in the OIR model.

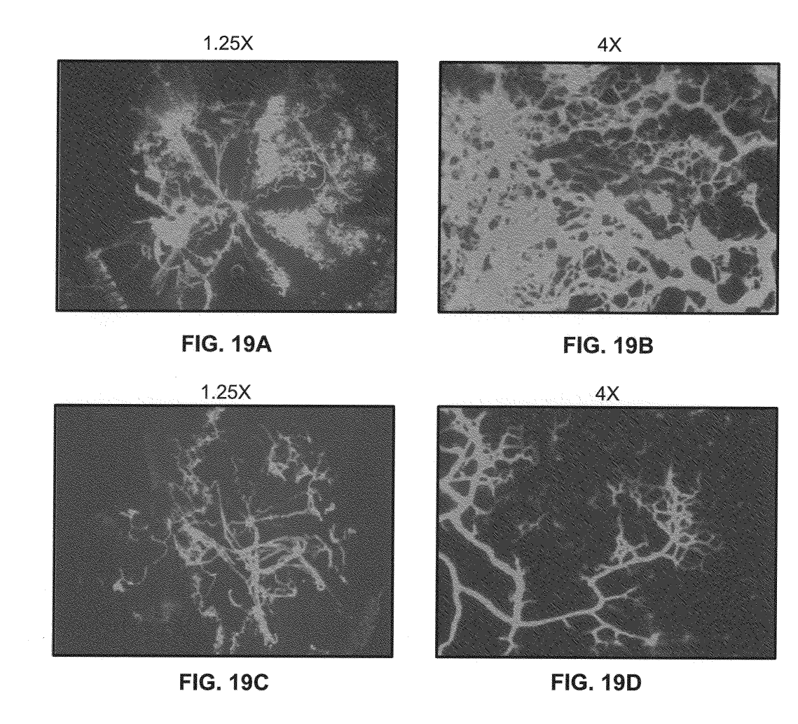

According to embodiments of experimental data and as shown in FIGS. 19A-19D, the OIR model was generated by exposure of BN rats to 75% oxygen for 5 days (P7-P12). At age of P15, OIR BN rats received a single intravitreal injection of DKK1 into the right eye and the same amount of BSA into the left eye. At P18, retinal vasculature was visualized using fluorescein angiography on the whole-mounted retinas from the left eye injected with BSA (FIGS. 19A and 19B) and the right eyes injected with DKK1 (FIGS. 19C and 19D) by angiography at P18.

### 19. DKK1 Inhibits Pre-Retinal NV in OIR Rats

Because pre-retinal NV (vasculature growing into the vitreous space) is a characteristic change of diabetic retinopathy, the effect of DKK1 was evaluated on pre-retinal NV. DKK1 was injected into the right vitreous of the OIR rats and BSA into the left vitreous at P15. At P18, pre-retinal NV was examined in retinal sections. The DKK1-injected eyes showed significantly fewer neovascular endothelial cells (EC) in the vitreous space than the contralateral eyes treated with the same amount of BSA. Furthermore, the nuclear translocation and expression levels of HIF-1α, a key transcription factor activating VEGF expression, were decreased in the retina of the DKK1-injected eye, compared to the eye treated with BSA. These results suggest that a single DKK1 injection partially arrests the pre-retinal NV in the OIR model via blocking the HIF-1 activation in the OIR model.

According to embodiments of experimental data and as shown in FIGS. 20A-20C, Brown Norway (BN) rats were exposed to hyperoxia for 5 days (P7-P12). At age of P15, the rats received a single injection of 3 μg of DKK1 into the vitreous of the right eyes and BSA into the left eyes. The eyes were sectioned at P18. Representative retinal sections from normal control rats (FIG. 20A), eyes of OIR rats injected with BSA (FIG. 20B), and OIR retina injected with DKK1 (FIG. 20C) were triple stained with three antibodies: Retinal vascular EC was stained with an antibody against CD31 (Red); the retinal ischemia stress status was detected by an antibody against GFAP (Blue), and the levels and nuclear translocation of HIF-1α were determined by an antibody against HIF-1α (Green).

### 20. DKK1 Reduces Retinal Vascular Leakage in Rats with STZ-Induced Diabetes

To investigate if blockade of the wnt pathway reduces the retinal vascular hyper-permeability in diabetes, 3 μg purified DKK1 was intravitreally injected into the right eyes of STZ-diabetic BN rats at 16 weeks following the STZ injection, and the same amount of BSA into the contralateral eyes as control. The vascular permeability was measured at 48 h following the DKK1 injection. As shown by vascular permeability assay, a single injection of DKK1 significantly reduced retinal vascular permeability in the STZ-induced diabetic rats.

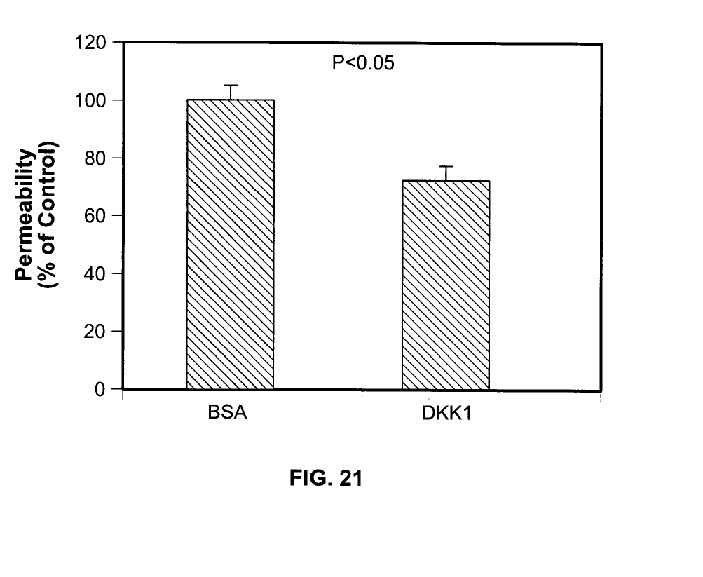

According to embodiments of experimental data and as shown in FIG. 21, DKK1 protein was injected into the vitreous of the right eyes (3 μg/eye) of STZ-diabetic rats at 16 weeks following the onset of diabetes, and the same amount of BSA into the contralateral eyes. The retinal vascular permeability was measured using Evans blue as a tracer, 48 hr after the injection. Vascular permeability was expressed as % of the permeability in the contralateral control (mean±SD, n=4), P<0.05.

### 21. Materials and Methods

Animals: Animals were maintained in a 12-h light/12-h dark cycle with an ambient light intensity of 85±18 lux at the cage level. Vldlr^{-/-} mice on the C57BL/6 background and wild-type (wt) C57BL/6 mice (The Jackson Laboratory, Bar Harbor, Me.) were used, treated and cared for in accordance with the statement for the Use of Animals in Ophthalmic and Vision Research set forth by the Association for Research in Vision and Opthalmology. Vldlr^{-/-} mice were genotyped following a PCR protocol recommended by The Jackson Laboratory.

Cell Culture: Human umbilical vein endothelial cells (HUVEC) were purchased from the American Type Culture Collection (Manassas, Va.). Cell culture reagents, fetal bovine serum, and chemicals were purchased from Invitrogen. ARPE19 cells were maintained in Dulbecco's modified Eagle's medium containing 3 mM L-glutamine, 10% fetal bovine serum, 100 units/ml penicillin G, and 100 μg/ml streptomycin sulfate at 37° C. in an environment containing 95% O₂ and 5% CO₂. HUVEC were cultured in endothelial cell basal medium (EBM-2, Cambrex, N.J.) maintained at 37° C. in an environment containing 95% O₂ and 5% CO₂ and supplemented with 5% fetal bovine serum, penicillin/streptomycin, and endothelial cell growth supplement (SingleQuots, Cambrex, N.J.). The cells were used in experiments between passage 4 and 6.

Fluorescein Angiography: Angiograms were performed using intracardiac injection of 10 mg/ml fluorescein isothiocyanate-conjugated high molecular weight dextran (Sigma, FD-2000S) in deeply anesthetized mice. Eyes were dissected and fixed with 4% paraformaldehyde in Hanks' balanced saline prepared immediately before use for overnight at 4° C., and retinas were flat-mounted in Fluoromount-G.

Immunohistochemistry: Eyes were enucleated and fixed in 4% paraformaldehyde overnight at 4° C. Cross-sections (5 μm) were cut and mounted on slides. To reduce autofluorescence background levels, the sections were blocked with 2% mouse serum and 10% normal goat serum in phosphate-buffered saline with 0.3% Triton X-100 for 1 h. Sections were stained with primary antibodies specific for VEGF (Santa Cruz, Calif.), β-catenin, GSK-3β, phosphorylated GSK-3β, and phosphorylated β-catenin (Cell Signaling, Danvers, Mass.), LRP5/6 (ABCAM, Cambridge, Mass.), CD31 (BD Pharmingen), and a rabbit anti-RDH10 antibody. Retinal sections were incubated with the primary antibodies for 1 h and washed thoroughly with phosphate-buffered saline. Secondary antibodies were added and incubated with the sections for 1 h. The sections were finally washed in phosphate-buffered saline and mounted in Fluoromount-G.

VEGF ELISA: The human VEGF QuantiGlo ELISA kit (R&D Systems, Inc., Minneapolis, Minn.) was used to measure VEGF levels in HUVEC and ARPE19 cells, and the mouse VEGF Quantikine ELISA kit (R&D Systems, Inc.) was used for mouse tissues following the manufacturer's protocol. The samples of the culture medium were concentrated 10 times, and the samples from mouse tissues were diluted 10 times to ensure that the VEGF concentration fell within the range of the VEGF standard curves.

Western Blot Analysis: The same amount (100 μg) of total proteins from mouse eyecups were used for Western blot analysis using specific primary antibodies for each protein and blotted with an horseradish peroxidase-conjugated secondary antibody. The signal was developed with a chemiluminescence detection kit (ECL; Amersham Biosciences). Blots were then stripped and re-blotted with an antibody specific for β-actin. Each protein band was semiquantified by densitometry and normalized by β-actin levels in the same gel.

Quantitative Real-time Reverse Transcription (RT)-PCR: Mice eyes were enucleated immediately after death into chilled diethylpyrocarbonate-treated normal saline, and the retinas were dissected. Total RNA was isolated using a commercial kit (Qiagen, Santa Clarita, Calif.). Primers (VEGF forward and VEGF reverse) were designed from the cDNA sequences spanning >1-kb introns using the Primer3 software. Total RNA (1.0 μg) was used for RT reactions, and 1 μl of the RT product and 3 pmol of primers were used for realtime PCR with a SYBR Green PCR Master Mix (Applied Biosystems). Fluorescence changes were monitored after each cycle. Amplicon size and reaction specificity were confirmed by 2.5% agarose gel electrophoresis. All reactions were performed in triplicate. The average C_{T} (threshold cycle) of fluorescence unit was used to analyze the mRNA levels. The VEGF mRNA levels were normalized by 18 S ribosomal RNA levels. Quantification was calculated as follows: mRNA levels (percent of control)=2Δ(ΔC_{T}), with ΔC_{T}=C_{T, VEGF}−C_{T, 18 S}, and Δ(ΔC_{T})=ΔC_{T,wt sample}−ΔC_{T,Vdlr}_{^{−/−}} sample.

Injection of Neutralizing Antibodies Specific for VEGFR2 and VEGFR3: Purified neutralizing antibodies for VEGFR2 and VEGFR3 (generous gifts from ImClone System) were separately injected into the subretinal space of Vldlr^{-/-} mice at age of P12. The eyes were dissected at P21 and fixed for NV analysis.

Transfection of Small Interference RNA (siRNA): The Cy3-labeled siRNA targeting VLDLR was commercially purchased from Ambion (Austin, Tex.). Transfection was performed using siPORT Amine (Ambion) following the instructions of the manufacturer. Briefly, 5×10⁶ HUVEC were incubated with the transfection mixtures containing 100 pmol of the Cy3-labeled siRNA for VLDLR or a Cy3-labeled control siRNA with a scrambled sequence for 24 h at 37° C. in 5% CO₂. Twelve hours after the transfection, the cells were washed twice with phosphate-buffered saline to remove transfection mixtures and cultured in Dulbecco's modified Eagle's medium containing 5% fetal bovine serum until they were used.

Subretinal Injection of Purified Mouse Dickkopf-1 (DKK1) Protein: Purified DKK1 (R&D System, MN) was injected into the subretinal space of the right eye (5 μg/eye), and the same amount of bovine serum albumin (BSA) was injected into the left eye of Vldlr^{-/-} mice at age of 4 weeks. Eyeballs were harvested 24 h after the injection, and the eyecups were dissected for analysis.

Intravitreal injection: All solutions will be sterilized by filtration and assessed for endotoxin. Animals will be anesthetized, and compounds will be injected into the vitreous of the one eye through the pars plana using a Hamilton syringe. The left eye will receive the same volume of vehicle and will be used as the control. Following injection, the animals will receive equal amounts of topical antibiotic ointment on both eyes. The animals will then be kept in normoxic conditions until the necessary time point for evaluation

Measurement of vascular permeability: Vascular permeability will be quantified by measuring FITC-BSA leakage from blood vessels into the retina following a method with modifications. The mice anesthetized and FITC-BSA (10 mg/kg body weight) is injected through the femoral vein under microscopic inspection. After injection, the mice are kept on a warm pad for 3 h to ensure the complete circulation of FITC-BSA. Then the chest cavity is opened, blood is collected through right atrium. Mice are perfused via the left ventricle to remove unbound dye with 1×PBS (pH 7.4), which is pre-warmed to 37° C. to prevent vasoconstriction. Immediately after perfusion, the eyes are enucleated, and the retinas are carefully dissected under an operating microscope. The fluorescein-albumin is extracted by sonication and centrifugation. The fluoresce density of fluorescein-albumin from supernatant and serum is measured at excitation wave 485 nm/emission wave 530 nm. Retinal protein levels are measured in the pellet by Bradford assays with quantification at A280. The FITC-BSA levels in the retina are then calculated by the supernatant fluoresce density and normalized to retinal protein levels and normalized to serum FITC levels.

Statistical Analyses: All assays utilizing quantification will be subjected to rigorous statistical testing. Significance will be determined using one-way analysis of variance and the appropriate post-hoc tests using Bonferroni's pairwise comparisons (Prism, version 3.02; GraphPad).

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1: A method comprising:
providing a composition comprising a DKK-related composition to treat a patient having an eye-related disease;
wherein a symptom of the eye-related disease is at least one of angiogenesis, inflammation, vascular leakage, and neovascularization.

2: The method of claim 1, wherein the eye-related disease is age-related macular degeneration.

3: The method of claim 1, wherein the DKK-related composition is DKK1.

4: A method comprising:
providing a composition comprising an antibody directed against a LRP 5/6 or VLDLR receptor to treat a patient having an eye-related disease;
wherein a symptom of the eye-related disease is at least one of angiogenesis, inflammation, vascular leakage, and neovascularization.

5: The method of claim 4, wherein the eye-related disease is age-related macular degeneration.

6: A method comprising:
providing a composition comprising a small interfering RNA directed against a LRP5/6 or VLDLR receptor mRNA to treat a patient having an eye-related disease;
wherein a symptom of the eye-related disease is at least one of angiogenesis, inflammation, vascular leakage, and neovascularization.

7: The method of claim 6, wherein the eye-related disease is age-related macular degeneration.

8: A method comprising:
providing an agent to treat an eye-related disease that interferes with the wnt pathway effecting increased phosphorylation of β-catenin;
wherein a symptom of the eye-related disease is at least one of angiogenesis, inflammation, vascular leakage, and neovascularization.

9: The method of claim 8, wherein the agent interferes with the wnt pathway by preventing binding of ligands to a wnt pathway receptor.

10: The method of claim 9, wherein the wnt pathway receptor is LRP5/6.

11: The method of claim 10, wherein the agent is an antibody against an LRP5/6 or VLDLR receptor.

12: The method of claim 8, wherein the agent comprises a small interfering RNA complementary to LRP5/6 or VLDLR receptor RNA.

13: The method of claim 10, wherein the agent is a DKK-related composition.

14: The method of claim 13, wherein the DKK-related composition is DKK1.

15: The method of claim 8, wherein the eye-related disease is age-related macular degeneration.

16: A method comprising:
providing an agent to treat an eye related disease that modulates LRP5/6 activity by preventing binding of other molecules to the LRP5/6 receptor;
wherein a symptom of the eye-related disease is at least one of angiogenesis, inflammation, vascular leakage, and neovascularization.

17: The method of claim 16, wherein the agent is a DKK-related compound.

18: The method of claim 17, wherein the DKK-related compound is DKK1.

19: The method of claim 10, wherein the agent is an antibody against an LRP5/6 or VLDLR receptor.

20: The method of claim 16, wherein the agent comprises a small interfering RNA complementary to LRP5/6 or VLDLR receptor RNA.

21: The method of claim 16, wherein the eye-disease is age-related macular degeneration.
